(19) United States
(12) Patent Application Publication
(10) Pub. No.: US 2011/0244500 A1
(43) Pub. Date: Oct. 6, 2011

(54) Gel electrophoresis method useful for resolution and characterization of nerve tissue ultra high molecular weight protein aggregates

(76) Inventor: Howard K. Shapiro, Narberth, PA (US)

(21) Appl. No.: 13/065,886

(22) Filed: Apr. 1, 2011

Related U.S. Application Data
(60) Provisional application No. 61/341,520, filed on Apr. 1, 2010.

(51) Int. Cl. C12Q 1/37; C07K 14/00

(52) U.S. Cl. 435/23; 530/350; 204/461

(57) ABSTRACT

The instant disclosure describes an electrophoretic procedure capable of resolving and isolating ultra high molecular weight (MW) protein aggregates from nerve tissue. The procedure is based on the use of composite agarose-polyacrylamide gel electrophoresis (CAPAGE) and resolves proteins and protein aggregates over the range of from approximately 225 kDa to approximately 30,000 kDa. Triton X-100 precipitation is used to obtain a cytoskeleton protein fraction that is subsequently resuspended and subjected to gel electrophoresis. This method demonstrates that a protein aggregate of approximately 30,000 kDa is characteristic of normal murine spinal cord tissue and that the amount of said protein aggregate is increased in spinal cord homogenate obtained from transgenic mice bearing copies of a mutant human gene characteristic of familial amyotrophic lateral sclerosis. This method for separating nerve tissue ultra high MW cytoskeleton protein aggregates can prove useful in a variety of future biophysical and pharmacological studies related to the etiologies of Charcot-Marie-Tooth disease, Alzheimer's disease, Parkinson's disease, diseases based on expansions in tandem DNA repeats, spinal muscular atrophy, Friedreich's ataxia, giant axon neuropathy, juvenile ceroid-lipofuscinosis, amyotrophic lateral sclerosis, diabetic polyneuropathy and Down's syndrome.

### RELATED PATENT APPLICATION

This invention is a continuation-in-part of U.S. Provisional Patent Application 61/341,520, filed on Apr. 1, 2010, entitled “Gel Electrophoresis Method Useful for Resolution and Characterization of Nerve Tissue Ultra High Molecular Weight Protein Aggregates,” now pending, the disclosure of which is incorporated by reference herein.

### BACKGROUND OF THE INVENTION

1. Field of the Invention

The present invention defines a new laboratory research method useful for resolution and biophysical characterization of nerve tissue ultra high molecular weight protein aggregates. This new method is useful for examining some aspects of the etiology of familial or non-familial neurodegenerative diseases selected from the closed group hereby limited solely to Charcot-Marie-Tooth disease, Alzheimer's disease, Parkinson's disease, diseases based on expansions in tandem DNA repeats, spinal muscular atrophy, Friedreich's ataxia, giant axon neuropathy, juvenile ceroid-lipofuscinosis, amyotrophic lateral sclerosis, diabetic polyneuropathy and Down's syndrome, including experimental animal models of said human diseases. For purposes of this disclosure, diseases based on expansions in tandem DNA repeats shall refer to the closed group limited to Huntington's disease; Kennedy's disease also known as bulbar and spinal muscular atrophy; dentatorubral-pallidoluysian atrophy; and spinocerebellar ataxia types 1, 2, 3, 6, 7 and 17. For purposes of this disclosure, each of the familial, i.e., genetic, neurodegenerative diseases within the field of the invention shall include any genetic variations thereof, i.e., genetic polymorphisms thereof. For purposes of this disclosure, experimental animal models of said human diseases shall include transgenic non-human animal models corresponding to a previously stated human neurodegenerative disease and gene knockout non-human animal models corresponding to a previously stated human neurodegenerative disease.

Insofar as the diminution of said nerve tissue ultra high molecular weight protein aggregates that characterize in part said neurodegenerative diseases can be regarded as an improvement in the health status of animals representing experimental models of said human neurodegenerative diseases, the instant disclosure can have utility in laboratory research protocols suitable for use in screening of candidate therapeutic drug agents. Said utility can be in the form of observing, i.e., comparing to one another, the optical densities of two or more ultra high molecular weight protein aggregates on a composite agarose-polyacrylamide gel, so as to partially characterize the molecular events occurring in the study of a candidate therapeutic drug agent. The present invention also provides the opportunity to obtain nerve tissue ultra high molecular weight protein aggregate metabolic markers characteristic of the presence of said neurodegenerative diseases, which can be used to monitor the pathological, i.e., clinical, stage of one or more said disease and can be used to provide metabolic markers related thereto. The present invention also provides the opportunity to study the biophysical composition of animal or human nerve tissue ultra high molecular weight protein aggregates obtained at autopsy.

2. Description of Prior Art

Several neurodegenerative diseases are characterized in part by the appearance of protein aggregates. The number and increasing size of such aggregates are generally regarded as being cellular hallmarks of disease progression. For example, neurofibrillary tangles and amyloid plaques are intracellular and extracellular markers for Alzheimer's disease (AD), respectively; Lewy bodies are cellular markers for Parkinson's disease (PD); axonal spheroids are cellular markers for amyotrophic lateral sclerosis (ALS); and aggregates consisting largely of mutant expanded polyglutamine repeat forms of huntingtin are characteristic of Huntington's disease. The appearance of such protein aggregates has been actively studied in recent years, work that has predominantly involved the application of immunohistochemical and molecular biological methods. Yet, while such aggregates have lent themselves well to productive studies by these approaches, their size and heterogeneous molecular composition make them poor subjects for study by conventional methods of analytical protein chemistry. For the most part, such aggregates appear to consist of oxidized, covalently cross-linked nerve cell cytoskeleton proteins, as well as other proteins. By microscopic examination, these aggregates appear to vary over a wide size range, with larger aggregates more characteristic of later pathological stages. Also, a variety of biophysical studies have attested to their generally hydrophobic nature. This creates problems related to buffer solubility, as well as spurious adhesion to glass/plastic surfaces, chromatography column matrices and filtration membranes. They cannot be resolved by conventional sodium dodecyl sulfate/polyacrylamide get electrophoresis (SDS/PAGE) and hence no Western blot method useful for their analysis has been described. Much remains to be done in order to define new analytical procedures useful for the study of proteins larger than 1,000 kDa. The instant disclosure addresses these deficiencies in the prior art and describes methods for overcoming them.

The presently described method allows for the isolation of nerve tissue cytoskeleton ultra high molecular weight (MW) protein aggregates by use of composite agarose-polyacrylamide gel electrophoresis (CAPAGE). This method is an adaptation of earlier CAPAGE protocols used to study other biochemical subjects that feature high molecular weight proteins. Such subjects include, for example, the study of proteoglycans (Melrose et al. 2000, Knox et al. 2001), lipoproteins (Gabelli et al. 1986) and blood platelet multimeric proteins (Counts et al. 1978, Hayward et al. 1991). The procedures used in these earlier studies include generally similar, but not identical, forms of CAPAGE. Distinctions among them can be found that relate to methods of gel preparation, grades of agarose, final percentage of agarose, ingredients used in preparation of polyacrylamide, final percentage of polyacrylamide, sample buffer composition, gel buffer composition, electrophoresis buffer composition, electrophoretic power supply conditions, methods of gel fixation, and methods of gel staining. But while many such distinctions can be found in the literature, various forms of CAPAGE methodology have much in common. They provide a form of gel electrophoresis which lends itself well to the study of ultra high MW proteins and protein complexes, they overcome the problems characteristic of SDS/PAGE gels having less than 5% polyacrylamide, they avoid the brittle nature of conventional agarose gels, they provide the opportunity for protein transfer onto nitrocellulose or synthetic membrane sheets for conventional Western blot analysis, and they can be used for in-gel Western blot analysis (see below DETAILED DESCRIPTION OF THE INVENTION, Section 2). However, said procedures used in these earlier studies were never uniquely adapted and used for the purposes disclosed herein.

Only brief descriptions of CAPAGE methodology have appeared in the literature (Andrews, 1981; Righetti, 1989). Composite agarose-polyacrylamide gels are not commercially available. Previous investigators have provided varying degrees of explanation for exactly how they have made and used such electrophoresis gels, and the present inventor is unaware of any previously disclosed step-by-step procedure of this kind. Issues that must be addressed include the innate contradiction between the need to keep agarose solutions heated in order to keep them liquid and the fact that solution heating greatly accelerates the polymerization of acrylamide. This physical conundrum makes the pouring of such composite thin layer electrophoresis gels problematical. Also, previous investigators have noted problems related to the removal of Teflon well combs, once gels have polymerized (Heinegard et al. 1985, Heimer et al. 1987, Varelas et al. 1991). The instant disclosure provides practical answers to such methodological problems in the form of laboratory techniques developed by this inventor that were not obvious to one of ordinary skill in the art. The instant disclosure of CAPAGE methodology describes both the extension of its utility to the study of nerve tissue ultra high MW protein species and provides sufficient methodological detail so as to provide a practical tool for others who may be interested in pursuing similar studies of their own. This procedure may serve as a useful tool for the investigation of nerve protein aggregation in human neurodegenerative diseases and animal models thereof.

Some reference to earlier studies can serve to better define the utility and novelty of the presently disclosed invention. In the absence of a practical method for isolating ultra high MW nerve protein aggregates, investigators have resorted to alternative investigative approaches. For example, Diaz-Hernández and coworkers (2004) described the use of discontinuous sucrose density centrifugation to isolate “the aberrant proteinaceous aggregates found in the brain of HD patients” (page 9368). Yet, compared to the presently disclosed CAPAGE procedure, their approach is cumbersome and apparently flawed in some respects. For each sample, their aggregate isolation procedure first required doing three centrifugation steps, and then doing a discontinuous sucrose density gradient centrifugation. The contents of the gradient fractions were then characterized by conventional 10% SDS/PAGE (their FIG. 2-A). In their SDS/PAGE work, they could only describe protein aggregate formation by reference to a band at the stacking gel/resolving gel interface. This says nothing about the size of such aggregates or how heterogeneous such aggregates might be, in contrast to the instant disclosure.

In addition, their presentation appears to be flawed in three respects. First, in their FIG. 2-A, they presented a flow chart of their protein isolation procedure that included the definition of centrifugation pellet fractions P1, P2 and P3. Then in FIGS. 2-B, 2-C and 2-D they presented Western blot and stained gel images describing the analysis of their various fractions. However, in these gel images reference to pellet P1 does not appear. Second, while they presented gel images that included the stacking gel/resolving gel interface and used the band at that position as an indicator of high MW protein aggregate formation, they failed to include the sample loading wells in their figures (e.g., FIGS. 2-B, 2-C, 2-D and 5-E). The formation of protein aggregates larger than those trapped at the stacking gel/resolving gel interface would have led to the retention of ultra high MW protein aggregates in the loading wells. Yet no such information was presented. In a third limiting aspect of their study, Diaz-Hernández and coworkers failed to recover any material that might have sedimented to the bottom of their sucrose density gradients. In work of this kind, it is only reasonable to expect that the largest protein aggregates will sediment through the entire gradient and collect on the bottom of the tube as a thin film. Such a film might not be noticed by the investigator, but the possible presence of such ultra high MW protein aggregates is a question that should be considered.

The observations of Diaz-Hernández and coworkers may describe one of two possible classes of huntingtin protein aggregates. They have described a form of protein aggregate that is loosely held together and relatively small in size (i.e., trapped at the stacking gel/resolving gel interface). They presented microscopy images of such aggregates in FIGS. 3, 4 and 6 of their report. However, by failing to describe the composition of pellet P1; by failing to reveal what, if any, protein was retained in their gel sample loading wells; and by failing to address the question of whether any protein aggregate migrated to the bottom of their sucrose density gradients, they failed to capture a view of any larger, more cross-linked protein aggregates that might have been present.

In their discussion, Diaz-Hernández and coworkers stated that “Here we report, for the first time, a protocol to isolate the aberrant proteinaceous aggregates found in the brain of HD patients” (page 9368). Yet, insofar as their statement might be construed as an indication that they had isolated ultra high MW aggregates of mutant HD, their statement appears to be a misinterpretation of their findings. In fact, the normal HD peptide monomer has a MW of ˜350 kDa, and any aggregate of that monomer would have a size in the range of >1,000 kDa. Based on use of conventional 10% SDS/PAGE, Diaz-Hernández and coworkers reported that they had isolated human HD post mortem protein aggregate at the sucrose gradient 20%-50% interface that consists of monomeric peptides of about 50 kDa (their FIG. 7-C), which they equate to similar material isolated by the same procedure from brains of transgenic mice (their FIGS. 2-B and 2-C) having only a mutated HD exon 1 fragment (the Tet/HD94 strain of Yamamoto et al. 2000). Hence, the Tet/HD94 mouse data reported by Diaz-Hernández and coworkers only relates to an exon 1 model of full length HD, their human sample analysis appears to define no more than proteolytic fragments of mutant HD (see Lunkes et al. 2002), and nothing in the reported work of Diaz-Hernández and coworkers indicates that they ever isolated intact aggregates of human mutant HD. Reports of this kind should not be confused with true isolation of the ultra high MW protein aggregates characteristic of diseases such as HD, other polyglutamine repeat disorders, ALS, AD or PD.

The work of Liao and coworkers (2004) also illustrates the present state of the art concerning nerve protein aggregate analysis. These investigators used liquid chromatography coupled with tandem mass spectrometry to study the composition of Alzheimer disease amyloid plaques obtained by use of laser capture microdissection. They described their work as “a comprehensive proteomic analysis of senile plaques from postmortem AD brain tissues.” Yet their study appears to have limitations at the conceptual level, at the methodological level, and at the level of data analysis.

At the conceptual level, there is an apparent contradiction between the use of 6%-12% SDS/PAGE gels to separate polypeptides in the size range of ˜10-300 kDa when these polypeptides were extracted from senile plaque protein aggregates large enough to be seen by use of fluorescence microscopy. The constituent polypeptides were extracted by two cycles of heating to 65° C. for 15 minutes with samples immersed in lysis buffer (pH 7.2 phosphate buffered saline [PBS] with 2% SDS, 10% glycerol, 10 mM dithiothreitol [DTT], 1 mM ethylenediamine-tetraacetic acid [EDTA] and Roche Applied Science protease inhibitor mixture). Yet it is well known that AD senile plaques consist of various proteins that are bound together covalently. Based largely on immunohistochemical studies, at least five forms of covalent protein cross-linking have been associated with AD senile plaques. These classes are (1) malondialdehyde-mediated cross-linking (Dei et al., 2002), (2) 4-hydroxynonenal-mediated cross-linking (Liu et al., 2003), (3) reducing sugar-mediated cross-linking (Vitek et al., 1994), (4) tyrosine dimer-mediated cross-linking (Atwood et al., 2004), and (5) transglutaminase-mediated cross-linking (Appelt and Balin, 1997; Norlund et al., 1999; Singer et al., 2002). Covalently cross-linked proteins are not solubilized by treatment with SDS and/or DTT. In reading the report of Liao and coworkers, one must suppose that somehow the covalently cross-linked proteins of AD senile plaques have become broken down or solubilized into their original native protein components. But Liao and coworkers provide no explanation of how this can happen.

At the methodological level, it is difficult to understand why Liao and coworkers used 6%-12% SDS/PAGE gels to study proteins in the MW range of ˜10-300 kDa. This PAGE system is routinely used to study soluble proteins, not proteins bound up in extremely large aggregates. So what happened to the preponderance of the protein aggregates that Liao and coworkers studied? Such ultra high MW aggregates might have suffered one or more fates. The ultra high MW aggregates may have stacked up as a very thin film at the bottom of their gel loading wells. If they used stacking gels over their 6%-12% PAGE gels, than aggregates might have also accumulated as a narrow band at the stacking gel/resolving gel interface. Alternatively, their ultra high MW protein aggregates may have never made it into the loading wells. As senile plaques are hydrophobic, such protein aggregates can be expected to adhere to glass or plastic surfaces, in which case they may have been lost prior to the addition of samples into gel loading wells. The use of siliconized utensils might have minimized such surface retention losses, but Liao and coworkers make no reference to having taken such precautionary measures.

At the level of data analysis, the report of Liao and coworkers also left some questions unresolved. They sliced each gel lane into fifteen segments and then analyzed these individually by liquid chromatography coupled with tandem mass spectrometry. This should have permitted the presentation of protein mass spectrometry data in comparison to the gel electrophoresis images of their FIG. 1-B, which shows stained gel lane bands and a kDa MW scale. But Liao and coworkers did not do so. Liao and coworkers obtained the raw tandem mass spectrometry data for such correlations to gel band positions, but never identified where their identified proteins actually migrated on the gels. For any particular protein listed in their Table I, they never explained if it was present as a single discrete band, if it was present in multiple bands or if it was present as a smear spread down a gel lane. Were their proteins just stacked up at the bottoms of loading wells? Such information was not provided. Looking at their FIG. 1-B, it is also curious that the only obvious difference between the stained protein bands in their AD senile plaque gel lane and the control gel lane is an AD lane band at 13 kDa.

Much, if not all, of the reported work of Liao and coworkers might be explained on the following grounds. It seems that their data represent proteins that were loosely and non-covalently bound to AD senile plaques. Such proteins would have been carried through their procedure, solubilized by SDS/DTT and successfully resolved on 6%-12% PAGE gels. These would have been the proteins at the edges of plaques, but not yet covalently cross-linked thereto. Although the title and text of their paper suggest that they comprehensively analyzed the content of established senile plaques, a careful examination of their report indicates that their method of constituent protein extraction/solubilization and their method of gel electrophoresis would not have permitted this.

As exemplified by the reports of Diaz-Hernández and coworkers and Liao and coworkers, methodological limitations have continued to impede the study of ultra high MW protein aggregates that are the cellular hallmarks of several neurodegenerative diseases. The present inventor demonstrates herein that CAPAGE methodology can be adapted for the analysis of such protein aggregates as found in normal mouse spinal cord tissue or in spinal cord tissue obtained from mice having a transgenic model of ALS. By employing this approach, a variety of future biophysical and pharmacological studies on such protein aggregates and their corresponding pathological equivalents in other neurodegenerative disease states can be envisioned.

Agarose gel electrophoresis is a well-recognized method for resolution of cerebrospinal fluid IgG molecular species characteristic of multiple sclerosis and other demyelinating disorders (Lubahn and Silverman, 1984). The present inventor is aware of only one report that describes the use of CAPAGE in studies on a protein found in nerve tissue. This is the study of Maeda et al. (1991) on the mouse cerebellum inositol 1,4,5-triphosphate receptor channel. Maeda and coworkers used 0.5% agarose/1.75% polyacrylamide gels to show that the functional inositol 1,4,5-triphosphate (InsP₃) receptor channel is a complex of four noncovalently bound identical peptides, each of ˜320 kDa in size. They used bis(sulfosuccinimidyl)suberate to cross-link InsP₃ receptor channel monomers, demonstrating the formation complexes having from two to four peptide subunits. They also used other chemical cross-linking agents to demonstrate the presence of the same four-unit complex in microsomes prepared from mouse cerebellar homogenates. So, some precedent exists for the use of CAPAGE gels in the analysis of nerve tissue proteins. But this approach has not previously been extended to the study of ultra high MW cytoskeleton protein aggregates characteristic of normal nerve tissue or nerve tissue samples characteristic of neurodegenerative disorders, and has not been extended to study protein aggregates across the molecular weight range of the instant disclosure.

### DETAILED DESCRIPTION OF THE INVENTION

As presently illustrated, this research methodology can be viewed as a “Rosetta Stone” for defining the nature of transgenic murine ALS high MW protein aggregates, and use of this methodology can be of value in the study of other animal models of human neurodegenerative diseases as well as the human disease states themselves (e.g., autopsy nerve samples).

### Section 1 Laboratory Method for Resolution of Nerve Ultra High Molecular Weight Protein Aggregates by Use of Composite Agarose-Polyacrylamide Gel Electrophoresis

The preferred embodiment of this method is disclosed as follows. Various modifications of this preferred embodiment may also be envisioned that still reside within the metes and bounds of this disclosure. Such modifications include the use of nerve tissue samples obtained from humans or other animals, use of alternative protein concentration assays, use of alternative grades of agarose, use of alternative concentrations of agarose, use of alternative grades of polyacrylamide, use of alternative concentrations of polyacrylamide, use of alternative buffers, use of gel electrophoresis equipment as an alternative to use of the Mini-PROTEAN II system of Bio-Rad, use of an electrophoresis power supply setting of other than constant 200 V, use of an alternative digital scanner to capture visual images of CAPAGE gels, and other minor variations in methodological details.

Another aspect of this invention as defined in this section provides a method that permits the isolation of ultra high molecular weight nerve protein aggregates of various sizes that can be subsequently used as new and novel disease-specific antigens. Said antigens can then be used to produce disease-specific monoclonal or polyclonal antibodies. Such antibodies can be used as the basis for a diagnostic test (e.g., a practical diagnostic test based on adapted use of prior art enzyme-linked immunosorbent assay [ELISA] technology), in tissue screening histological studies or to possibly identify the presence of sub-clinical amounts of disease-specific antigen(s) in patient blood or urine samples obtained from patients having any one of the closed group of neurodegenerative diseases addressed herein. The identification of one or more such disease-specific antigens in patient blood or urine samples can, in turn, provide the basis for simple, photometric, inexpensive clinical or home-use diagnostic assays capable of providing on-going physiological data and response to treatment on an on-going basis.

Materials. SeaKem Gold agarose (catalog no. 50152) and ProSieve 50 acrylamide gel stock solution (catalog no. 50618) were from BioWhittaker Molecular Applications. Chemiluminescent BlueRanger™ Protein Molecular Weight Marker Mix (catalog no. 26681), AquaSil Siliconizing Fluid (catalog no. 42799), BCA Protein Assay Reagent A (catalog no. 23223), BCA Protein Assay Reagent B (catalog no. 23224), tetramethylethylenediamine (TEMED) (catalog no. 17919), ammonium persulfate (AMP) (catalog no. 17874), glycerol (catalog no. 17904), and Surfact-Amps® X-100 (a 10% Triton X-100 solution in water, catalog no. 28314) were from Perbio Science. Sodium cyanoborohydride (NaCNBH₃), 95+% (catalog no. 16855-0100) was from Acros. Electrophoresis gel 200 μl sample loading micropipettor tips (catalog no. 02-707-138) were from Fisher Scientific. PTFE microbore tubing (ID 0.032″, OD 0.056″, catalog no. A-06417-31, not siliconized) was from Cole-Parmer Instrument Company. Ethylenediamine-tetraacetic acid (EDTA) (free acid, catalog no. ED), sodium azide (catalog no. S2002), DL-dithiothreitol (DTT) (catalog no. D9163), bovine serum albumin (catalog no. A7638) and Protease/Phosphatase Inhibitor Cocktail (catalog no. P8340) were from Sigma-Aldrich. Gel electrophoresis was performed using the Mini-PROTEAN II system of Bio-Rad. Additional Bio-Rad products used included 1.5 M Tris-HCl, pH 8.8 (catalog no. 161-0798), Tris/glycine/SDS10× buffer (catalog no. 161-0732) and 20% sodium dodecyl sulfate solution (catalog no. 161-0418). The 20% SDS solution was diluted to 10% by addition of deionized water. A Kontes Pellet Pestle tissue homogenizer (catalog no. K749540-0000) and 1.5 ml polypropylene pestles with matching microtubes (catalog no. K749520-0000) were from Fisher Scientific.

Only siliconized glassware (e.g., Pyrex no. 5580 tubes with red/white Teflon lids), homogenizer parts (i.e., Pellet Pestle 1.5 ml polypropylene pestles with matching microtubes) and other plasticware (e.g., micropipette tips) were used. Items to be siliconized were first rinsed twice in absolute ethanol and allowed to dry. Then AquaSil Siliconizing Fluid was used to siliconize these items. The AquaSil working solution was prepared by adding 500 μl of stock solution to 49.5 ml deionized water in a small plastic tray. Items were rinsed once in about 60-70 ml methanol (MeOH) in a second small plastic tray. Likewise, the Bio-Rad glass plates (catalog numbers 1652907 and 1652908) used for preparation of composite agarose-polyacrylamide gels were also siliconized. For each of the Bio-Rad glass plates, it is preferable to hold what will be the bottom edge with a hemostat and to vertically immerse the plate partly into AquaSil working solution in a beaker, taking care to not expose about 5 mm of glass on the intended bottom edge of the plate. Not coating the bottom 5 mm of each glass plate will help ensure that the 5 mm plug layer of 10% polyacrylamide gel will stay in place.

Animals. A colony of transgenic B6SJL-TgN(SOD1-G93A)1Gur mice (Jackson Laboratories) and their non-transgenic littermate controls was maintained in the laboratory of Dr. Terry D. Heiman-Patterson at Drexel University College of Medicine. For the data obtained and described in this section and in Section 2 below, mice were 148 days old at sacrifice, near the maximum longevity for transgenic mice of this kind.

Preparation of stock solutions. Mouse Spinal Cord Homogenization Buffer is Made as a 200 ml solution, and then stored in 25 ml aliquots at −20° C. The 200 ml solution is 50 mM NaPO₄ pH 7.4 and 5 mM EDTA to which 200 μl Sigma-Aldrich Protease/Phosphatase Inhibitor Cocktail is added. This is prepared as follows. To make 100 ml of 10 mM EDTA, free acid, weigh out 292.26 mg and add to 90 ml deionized water. Use concentrated NaOH (i.e., 50%) to raise pH to about 7.3. Then use dilute NaOH (i.e., N/20) to raise pH to 7.4. Bring the EDTA solution up to 100 ml, and then combine with 100 ml of 100 mM NaPO₄, pH 7.4 and 200 μl Sigma Protease/Phosphatase Inhibitor Cocktail.

The gel loading buffer is 25% glycerol (v/v) in 0.5 M Tris pH 6.8 with 100 mM DTT. Make 20 ml in a 50 ml glass graduated cylinder. To do so, start by adding 5 ml glycerol into the graduated cylinder. This is most accurately done by weight on a platform scale; add 6.25 grams glycerol to the graduated cylinder. Weigh out 308.6 mg of DTT into a plastic weighing dish. Use a plastic disposable transfer pipette to add a few ml of 0.5 M Tris pH 6.8 to the weighing dish and then transfer the DTT/Tris into the graduated cylinder. Repeat this transfer process several times until all of the DTT is in the graduated cylinder and the volume has been raised to 20 ml. Seal the graduated cylinder with Parafilm-M® and invert repeatedly until the DTT crystals are dissolved and a homogeneous solution is obtained. This solution is stored at −20° C. for up to 6 months.

Preparation of mouse spinal cord samples. This procedure describes the processing of one ALS spinal cord and one control spinal cord. Each spinal cord will be homogenized into a final volume of 2.0 ml homogenization buffer, divided into four 500 μl aliquots in standard siliconized 1.5 ml microfuge tubes. Dissected mouse spinal cords are stored at −80° C. in the absence of buffer until weighing and homogenization. As protein aggregates may be quickly lost due to adhesion onto glass or plastic surfaces, every step of this procedure is designed to avoid such loss.

Pre-weigh two Fisher Scientific catalog no. K749520-0000 polypropylene Pellet Pestle siliconized 1.5 ml microfuge tubes. Each pre-weighed empty Pellet Pestle microfuge tube is put into a 50 ml plastic screw cap tube and put into a −20° C. freezer. Likewise, each of two Pellet Pestle plastic pestles is put into a 15 ml screw cap tube and put into a −20° C. freezer. When ready to begin processing a spinal cord specimen, insert about the lower two inches of a 50 ml plastic screw cap tube containing a pre-weighed 1.5 ml microfuge tube into dry ice pellets. This will cool down each empty 1.5 ml microfuge tube without exposing it to carbon dioxide vapor.

In this procedure, each spinal cord is placed onto a glass plate on dry ice, and a scalpel is used to finely mince the specimen. Exposure of the tissue to dry ice vapor is to be avoided, as such exposure would acidify the tissue. Take a plastic bag, such as that used for 14 ml or 50 ml plastic screw cap tubes, and cut it open at both ends. Put this on a lab bench on top of several books and put one layer of dry ice pellets into the bag. The open ends of the plastic bag will face down, so carbon dioxide vapor will fall away from anything placed on top of the bag. Put an 8×8″ glass plate on top of the plastic bag over the dry ice. Put a piece of plastic wrap on top of the glass plate to prevent atmospheric water vapor from condensing directly onto the glass plate. After 30 minutes, remove the plastic wrap and put the first frozen spinal cord on the glass plate. Then mince it finely with a pre-cooled razor blade held by a hemostat, producing pieces of about 1-2 mm³. Use the razor blade and a metal spatula previously cooled to −20° C. to transfer the pieces of minced spinal cord into a pre-weighed dry ice-cooled Pellet Pestle microfuge tube. Then snap shut the microfuge vial lid, put the vial back into its 50 ml plastic screw cap tube and affix the cap thereof. Insert about the lower two inches of this 50 ml tube back into dry ice pellets in a bucket or tray. This will quickly freeze down each minced spinal cord sample in its 1.5 ml Pellet Pestle microfuge tube without exposure of the tissue sample to carbon dioxide vapor. After several minutes on dry ice, open the 50 ml screw cap tube, gently tap the sealed frozen microfuge vial onto the pan of a Mettler scale and re-weigh the vial. The weight difference is the wet weight of minced spinal cord in the vial. Then quickly put each microfuge vial back into its 50 ml plastic tube, re-cap the tube and put it back into dry ice pellets to cool it down again. Typically, from adult mice weighing 21-25 gm, spinal cord wet weights are found to be in the range of 84-88 mg.

In order to stabilize and preserve any Schiff base cross-links that may be present, homogenates are reduced with 50 mM NaCNBH₃ (Esterbauer and Cheeseman, 1990). Weigh out and immediately add 16 mg of NaCNBH₃ into 5 ml homogenization buffer (kept on ice). Then immediately homogenize the ALS specimen in a total of 2.0 ml of this buffer. As described below, each 2 ml homogenate will be divided into four 500 μl aliquots. After Pellet Pestle homogenization, the four 500 μl aliquots derived from each spinal cord are then put onto a vertical wheel rotator for 4 hours at room temperature. This NaCNBH₃ homogenization procedure is then repeated for the control spinal cord, starting with the weighing out of 16 mg of NaCNBH₃ and its addition to 5 ml homogenization buffer.

Use of the Pellet Pestle homogenizer is summarized as follows. The motorized homogenizer is secured into a small metal clamp that is attached to a metal ring stand. The homogenizer is adjusted so that an attached plastic pestle will be in the vertical position, pointing down. A plastic pestle that was previously cooled to −20° C. is attached to the Pellet Pestle motor. The Pellet Pestle microfuge tube having the minced ALS spinal cord specimen is tightly secured into a second small metal clamp, so that the handle of the clamp can be held during homogenization. For the first of four homogenization steps, add 500 μl ice cold NaCNBH₃ homogenization buffer to the Pellet Pestle microfuge tube having the minced ALS spinal cord specimen.

Start mixing and continue plunging for about 2 minutes, until the suspension is mostly uniform and has a cloudy white appearance. It will still have some tiny visible tissue particles in it. Use a micropipettor with 1 ml micropipette tip to transfer 125 μl aliquots into four standard 1.5 ml microfuge tubes, which are kept on ice. The orifice of standard 200 μl micropipette tips is too small to permit their use. Then put each of the four 125 μl aliquot microfuge tubes into the small, metal clamp and mix again, each being mixed for about a minute. This helps grid up the tiny visible tissue particles, leaving apparently uniform cloudy suspensions. There is still a small amount of original homogenate in the Pellet Pestle microfuge tube, corresponding in microliters approximately to the milligram wet weight of the specimen.

To begin the second homogenization step, add to the original Pellet Pestle microfuge tube a volume of NaCNBH₃ homogenization buffer corresponding to 500 μl minus a volume equivalent to the specimen wet weight. So, for example, if the spinal cord weighed 84 mg, than add 416 μl NaCNBH₃ homogenization buffer. Mix again, and then transfer 125 μl aliquots to each of the four standard 1.5 ml microfuge tubes. All tubes are kept on ice, except when in the metal clamp for mixing. After the second 125 μl aliquots have been added to each of the four standard 1.5 ml microfuge tubes, put each of the four into the metal clamp and mix.

To begin the third homogenization step, add 500 μl fresh NaCNBH₃ homogenization buffer to the original Pellet Pestle microfuge tube, mix about 30 seconds, and then transfer 125 μl aliquots to each of the four standard 1.5 ml microfuge tubes. Do not put each of the four standard 1.5 ml microfuge tubes back in the metal clamp for mixing.

To begin the fourth homogenization step, add 500 μl fresh NaCNBH₃ homogenization buffer to the original Pellet Pestle microfuge tube, mix about 30 seconds, and then transfer 125 μl aliquots to each of the four standard 1.5 ml microfuge tubes. Put each of the four standard 1.5 ml microfuge tubes back in the handheld metal clamp for a final mixing. This protocol generates four virtually identical 500 μl aliquots of crude spinal cord homogenate. From each of these four samples, withdraw a 5 μl and a 10 μl aliquot for BCA protein assays, then store these aliquots at −20° C. Put the four microfuge tube homogenate samples onto a Glas-Col© vertical wheel rotator at speed setting 12 (i.e., slow rotation) for 4 hours at room temperature. This NaCNBH₃ homogenization procedure is then repeated for the control spinal cord specimen. After removal from the vertical rotator wheel, samples are stored at −80° C.

Cytoskeleton precipitation and resuspension by sonication. A crude cytoskeleton fraction is obtained by 0.5% Triton X-100 precipitation. Each sample is processed in a volume of 1 ml. The 10% stock solution of Triton X-100 is Perbio Science Surfact-Amps X-100. For each 500 μl of NaCNBH₃—reduced homogenate, use a micropipettor with a 1 ml micropipette tip to transfer the entire sample from its 1.5 ml microfuge tube into a Pyrex no. 5580 tube. Glass containers are best for bath sonication. Glass effectively transmits the oscillation, while plastic absorbs the oscillation. Put each Pyrex no. 5580 tube on ice. To bring each sample up to 1.0 ml at 0.5% Triton X-100, add 450 μl of homogenization buffer and 50 μl of the Surfact-Amps stock solution. Add half of the homogenization buffer (i.e., 225 μl) to the 1.5 ml microfuge tube as a rinse. After vortexing and momentary centrifugation in a microfuge to draw liquid away from the inside of the vial cap, add this rinse to the Pyrex tube. Repeat this rinse process with a second 225 μl aliquot of homogenization buffer. Then add 50 μl of Surfact-Amps stock solution to each Pyrex tube. Add a red/white Teflon lid which is manually held tight, vortex gently 10 times, invert 10 times, and then gently vortex 10 times. The lid/Pyrex tube joint must always be manually held tight, as the surfaces of both the siliconized Pyrex tube and Teflon lid are quite slippery. Leave each Pyrex tube on ice for at least 30 minutes, with occasional inversion and gentle vortexing.

Centrifuge Pyrex tubes at room temperature in a Sorvall RT 6000D centrifuge for 60 minutes at 3,500 rpm (2,100×g). By use of a swinging bucket rotor with green 35-position platform racks, precipitate collects at the bottom of each tube, not smeared up its side. After centrifugation, keep samples in a vertical position. For each, use a 1 ml micropipettor set on 500 μl with a standard 1 ml tip to aspirate off supernate once. Then put a flat-tipped gel well loading tip on a 200 μl micropipettor, position the flat tip on the glass wall just above the upper edge of the pellet, and carefully remove as much additional supernate as possible. Add each recovered supernate to a pre-weighed siliconized 1.5 ml microfuge tube. Seal each recovered supernate tube and then re-weigh it to determined the milligrams (i.e., microliters) of recovered supernate. Typically, the volume of each recovered supernate will be about 900 μl. Vortex each recovered supernate well, momentarily centrifuge in a microfuge to draw liquid away from the inside of the vial cap, then open the vial to obtain samples for BCA protein assays. From each vial, take three 10 μl and three 20 μl aliquots for protein assays. Store the recovered supernate samples at −80° C. and store the protein assay aliquots at −15° C.

For each Triton X-100 pellet, add 100 μl of gel loading buffer to its Pyrex tube. Then add the red/white Teflon lid and secure the lid into place with Parafilm®. Insert Pyrex tubes into a small plastic rack and then insert the plastic rack into a bath sonicator having only about 2 cm of water in its bath chamber. As such, the plastic rack is sitting on the bottom of the bath chamber, with a water level above the samples. For a Branson 1510 sonicator, use setting “set sonics min” and begin sonication. Sonicate samples for intervals of 10 minutes. After each 10 minute sonication, visually inspect the Pyrex tubes, strongly vortex them, and return them to the sonication bath. Such 10 minute intervals of sonication, followed by visual inspection and strong vortexing are repeated until both pellets appear to be completely resuspended by visual inspection. This takes from about 80 to 100 minutes of sonication. During sonication, the bath water is not being heated as a matter of intentional procedure, but sonication itself will slowly raise the water temperature to approximately 40° C.

Sonicated samples can be left for as long as 10 days at room temperature prior to gel electrophoresis. As such, they are denatured, detergent-coated, reduced proteins in gel loading well buffer. After bath sonication, it is preferable to not refrigerate or freeze them, as doing so might further contribute to protein aggregation. When sonicated samples are left at room temperature for more than 2 days, each of their subsequent gel well loading samples is brought up to 1% final SDS concentration and heated for 3 minutes at 97° C. immediately prior to electrophoresis.

BCA protein assays. Aliquots for BCA protein assays cannot be taken from the resuspended pellets because the gel loading buffer contains 100 mM DTT. BCA assays must therefore be done on the 5 μl and 10 μl aliquots of each spinal cord initial homogenate in homogenization buffer and on 10 μl and 20 μl aliquots of each supernate taken off a Triton X-100 pellet, followed by subtraction of the total supernate protein from the original homogenate total protein to determine the amount of protein in each resuspended Triton X-100 pellet.

As each Triton X-100 supernate is taken off it's Triton X-100 pellet, it is added into a pre-weighed siliconized 1.5 ml microfuge tube. Then each such siliconized microfuge tube is weighed again. Doing so allows for the determination of the weight of each recovered Triton X-100 supernate and hence it's exact total volume (i.e., assuming 1 mg equals 1 μl).

Based on BCA assays, calculate the total mg protein for each recovered Triton X-100 supernate. Then subtract that value from the total mg protein value for the crude homogenate used for the Triton X-100 precipitation. The difference is the amount of mg protein “passed on” in each Triton X-100 pellet to be added to the 100 μl of gel well loading buffer. After sonication of each Triton X-100 pellet in 100 μl of gel well loading buffer, record the volume of each aliquot thereof taken for addition to each gel lane loading well. Then use a micropipettor to take up whatever residual sonicated resuspended Triton X-100 pellet was left over after preparing all gel lane loading well samples. Record the size of each such residual sonicated resuspended Triton X-100 pellet that was left over. Taken together with the total of all aliquots taken for addition to gel lane loading well samples, the investor can accurately calculate the total volume of each sonicated resuspended Triton X-100 pellet. The investigator can then calculate how much protein was applied into each gel lane loading well.

BCA protein assays may be done either before or after gel electrophoresis. BCA assays are done in siliconized standard 1.5 ml microfuge tubes. The standard BCA protein assay procedure is used, but with one-half of all volumes and protein amounts. So, each assay includes 50 μl sample and 1 ml “working solution A +B” (i.e., from a stock solution consisting of 25 ml Reagent A and 0.5 ml Reagent B). Bring each of the 5 μl and 10 μl crude homogenate aliquots up to 50 μl with fresh homogenization buffer. Bring each of the μl and 20 μl Triton X-100 precipitation supernate aliquots up to 50 μl with a solution consisting of 950 μl homogenization buffer+50 μl of 10% Surf-Amps Triton X-100 stock solution. Two bovine serum albumin (BSA) stock solutions are prepared. One is 10 mg BSA in 10 ml homogenization buffer. The second is 10 mg BSA in 10 ml in homogenization buffer that contains 0.5% Triton X-100 (i.e., 950 μl homogenization buffer+50 μl of 10% Surf-Amps Triton X-100 stock solution). Store these at −20° C. in 1 ml aliquots. For each BCA standard curve, assay in duplicate 50 μg, 40 μg, 30 μg, 20 μg and 10 μg BSA. For BSA assays having less than 50 μg, bring each up to 50 μl with either homogenization buffer or a solution consisting of 950 μl homogenization buffer+50 μl of 10% Surf-Amps Triton X-100 stock solution. A BioMate 3 UV/visible spectrophotometer or equivalent thereof is used to record optical densities. Typically, each 2 ml spinal cord homogenate contains about 7.0-7.5 mg total protein.

Preparation of composite agarose-polyacrylamide gels. This method of CAPAGE gel preparation is a modification of the original work done by Peacock and Dingman (1968). Although a variety of agarose products are commercially available, use of SeaKem Gold agarose is preferred because of its high gel strength (≧1800 g/cm² for a 1% gel). SeaKem Gold has a relatively high melting point, requiring heating to about 90° C. Gels are poured at a thickness of 1.5 mm in Bio-Rad clamp assemblies for the Mini-PROTEAN II electrophoresis system. The combination of SeaKem Gold agarose and ProSieve 50 polyacrylamide produces gels having excellent physical characteristics. They retain their shape, yet they are flexible and can be handled much as one would handle a 7.5% standard SDS/PAGE gel. As many as six gels can be poured in sequence, using clamp assemblies on three casting stands. The procedure for preparing each gel is summarized as follows. There is no stacking gel. When the procedure is finished, each resolving gel is 0.5% agarose and 2.0% polyacrylamide, with a 5 mm layer of 10% polyacrylamide gel at its bottom. The 10% polyacrylamide layer serves a “plug” function, preventing the resolving gel from sliding down during electrophoresis. Prepare 300 ml of 2.0% agarose stock solution and, from this, 1.0% agarose is prepared as needed. In this procedure, equal volumes of 1.0% agarose and 4.0% polyacrylamide are briefly mixed and the gel is then quickly poured.

For preparation of the 5 mm “plug” 10% polyacrylamide layer, in a 50 ml plastic tube combine 1.35 ml deionized water, 500 μl ProSieve 50 stock solution and 625 μl 1.5 M Tris pH 8.8. Mix well and then add 1 μl of TEMED. Mix again and then add 25 μl of 10% AMP. Add 750 μl of this solution to the clamp assembly on a casting stand to obtain a 5 mm layer of poured gel. Then, slowly add about 7 ml deionized water, taking care to avoid mixing of the two layers. Let the gel polymerize for at least 30 minutes. Thirty minutes before the final steps of gel pouring, bring the casting stand with 10% polyacrylamide plug layer, a 10-well Teflon well comb and several plastic 10 ml pipettes into a 37° C. room. Immediately before the final steps of gel pouring, a 200 μl well loading pipette tip attached to a vacuum line is used to aspirate away the water over the 10% polyacrylamide plug gel.

In a 50 ml plastic tube, combine 3.075 ml deionized water, 0.6 ml ProSieve 50 and 3.75 ml 1.5 M Tris pH 8.8 (4.0% polyacrylamide solution). Using brief intervals of microwave heating, the 2.0% agarose stock solution is heated to 90° C. and mixed well. Add 3.75 ml of deionized water to a 50 ml plastic tube. Add 3.75 ml of the hot 2.0% agarose solution to the 3.75 ml of deionized water. The plastic cap is then loosely secured onto the 50 ml tube. The tube is then microwaved, so as to get the 1.0% agarose boiling and to raise its temperature to 85-90° C. To microwave the tube, put it into a 250 ml glass beaker and add tap distilled water up to about the 150 ml mark. Keep the heated agarose tube in the beaker water. On a laboratory bench, this solution is then allowed to cool slowly to 60° C., with its temperature carefully monitored.

The final steps of gel pouring are as follows: (1) when the 1.0% agarose stock is about 70° C., add 6 μl of TEMED to the 4.0% polyacrylamide solution and vortex; (2) when the 1.0% agarose is at 64° C., aspirate away the water layer from the 10% polyacrylamide plug gel in the 37° C. room; (3) add 75 μl AMP to the 4.0% polyacrylamide solution, then vortex momentarily; (4) when the 7.5 ml of 1.0% agarose is at 60° C., add it to the 7.5 ml of 4.0% polyacrylamide solution, then vortex momentarily; (5) in the 37° C. room, quickly draw about 12 ml of agarose/polyacrylamide solution into a 10 ml plastic pipette, then eject it back into its tube, and then draw about 12 ml back into the pipette (so as to achieve further mixing); (6) quickly pour the resolving gel by filling the gel slot of the clamp assembly up to the top of the small glass plate (adding about 9.6 ml); and then (7) quickly add the well comb. The well comb is added at a tilted angle on the right side of the gel casting slot, and then drawn across towards the left into a horizontal position. Doing so avoids the trapping of air bubbles. The gel will be starting to become viscous by the time the well comb is added, with jelly-like lumps of gel overflowing out of the gel slot. Steps (1)-(4) have been done on a laboratory cart outside of the 37° C. room, so that steps (5)-(7) can be done as quickly as possible. When two or more gels are being poured as a set, these final steps must be done on each gel one at a time.

Remove the gel from the 37° C. room. Use a spatula and vacuum line to remove excess gel from the clamp assembly. After 30 minutes at room temperature, add about 3 ml of deionized water to the horizontal well of the casting stand (to preserve humidity) and then place the clamp assembly on its casting stand with well comb still in place into a zip-lock plastic storage bag. Gels are stored at 4° C.

The correct method for removal of the well comb is as follows. The well comb cannot simply be pulled up. Nor can it be removed by first gently tilting it a bit clockwise and then counter-clockwise. Using either of these movements, some gel will continue to adhere to the more central well comb fingers. The well comb should be removed while the gel casting apparatus is still cold. Although unseen to the observer, for each loading well there is a thin film of gel between the Teflon finger of the well comb and each of the glass plates. This film adheres more firmly to the glass than to the Teflon surface, but the bond between the film and the Teflon must be broken before the well comb is removed.

Use a micropipettor to apply Bio-Rad 1× Tris/glycine/SDS running buffer along the well comb across both the front edge (with small glass plate) and the back edge, going all the way across the top of the small and large glass plates. The intent is to permit SDS to get down to the gel dividers between loading wells as soon as the well comb is wiggled. Holding the upper part of the well comb at its midsection, press it slowly forward (i.e., horizontally towards the investigator) and then push it away just a bit. This process is referred to as wiggling.

Then slowly pull the comb straight up ˜2 mm. Typically, the gel film of loading wells 1 and 10 will separate from the comb, but not so for the more interior wells. Slowly move the comb laterally from right to left ˜1 mm a few times, which should separate the Teflon comb fingers of loading wells 1 and 10. Then repeat the forward/back wiggling of the comb, doing so slowly and many times. Each time, more air pockets appear at the bottoms of loading wells, indicating that the gel is slowly letting go of the comb fingers. In the course of this wiggling, occasionally raise the comb straight up ˜1 mm. From the sides in towards the middle, more gel well dividers will detach from the comb. Also, occasionally move the comb latterly left and right slowly ˜1 mm, to further encourage the gel to detach from the comb fingers. Finally, it is obvious that all gel well dividers have detached from the comb, and the investigator can slowly finish raising the comb straight up.

Using a standard 200 μl well loading micropipette tip, straighten up the loading well gel dividers. It can now be seen that in most or all of the wells there is a thin film of gel going from one side of the well to the other, adhering to the glass plates. Use a 200 μl well loading pipette tip attached to a vacuum line to remove excess gel film from within the loading wells. If the electrophoresis gel is going to be used immediately, add 1× Bio-Rad electrophoresis running buffer up to the top of the small glass plate, filling the loading wells. If the gel is going to be used at a later date for up to 30 days, add deionized water up to the top of the small glass plate. For such storage, leave the gel assembly on its casting stand, place the apparatus into a plastic bag and store at 4° C. Every other day, use a micropipettor to restore the level of deionized water up to the top of the small glass plate, thus preventing gel dehydration. Alternatively, one may leave the well comb of one or more gels in place for several days or weeks before gel use. In doing so, a small amount of water evaporates from the sides of each well comb. Hence, every other day the upper corners of the gel should be rehydrated with several 5-20 μl aliquots of deionized water.

Gel loading and electrophoresis conditions. The gel clamp assembly of each gel that was stored at 4° C. is removed from its casting rack, set into a small plastic tray and allowed to come to room temperature. Tilting the gel assembly, water is removed from the sample loading wells by aspiration and the wells are rinsed twice with 1× Bio-Rad Tris/glycine/SDS running buffer. Running buffer is then added to the wells again and left there throughout the sample loading procedure.

If resuspended Triton X-100 pellet samples contain 1% SDS, than BlueRanger protein standard gel well loading samples are also brought up to 1% SDS. BlueRanger protein standard gel well loading samples should always contain an amount of sample loading buffer corresponding to the amount contained in each well loading sample having resuspended Triton X-100 pellet sample, although protein standard samples are never heated for 3 min at 97° C. prior to electrophoresis. In order to facilitate the visual observation of protein standards, three vials of BlueRanger protein mixture are loaded per lane.

Standard electrophoresis well loading micropipette tips (e.g., Fisher catalog no. 02-707-138 gel tips) cannot be used for the resuspended Triton X-100 pellet samples, because these samples are too viscous. In order to overcome this problem, well loading tips are modified as follows. For each micropipette tip, its orifice is slid into a piece of Cole-Parmer PTFE tubing (catalog no. A-06417-31) and secured by gently pressing the two together. This size of PTFE tubing has an internal diameter that is large enough to permit the accurate sampling of resuspended Triton X-100 pellet samples, while its 1.4 mm external diameter is just small enough to allow it to fit between the glass plates containing the electrophoresis gel. Using such a modified tip, a sample can be efficiently applied near the bottom of its loading well. By trial and error with water, an appropriate length of tubing can be selected. For example, 25 μl of resuspended Triton X-100 pellet sample can be taken up by a 5 cm length of tubing secured onto a standard well loading micropipette tip.

Shortly before loading samples into wells, a complete set of 10 well loading samples is prepared. Then these are quickly loaded and the electrophoresis run is initiated. Note: after aliquots have been withdrawn from a resuspended Triton X-100 pellet sample, it is important to determine the residual microliter volume still remaining in the tube. This may be done with a micropipettor, repeatedly drawing up the residual volume and re-setting the micropipettor as necessary. In the course of bath sonication to resuspend each Triton X-100 pellet, some volume is inevitably lost due to evaporation. In general, a sample will have about 200 μl volume at the start of bath sonication. Yet, after aliquots have been taken for gel loading well samples and the residual volume is measured, it can be calculated that the post-sonication total sample volume was in the range of 130 μl. This loss of volume must be taken into account when calculating how many micrograms of protein were actually added to each loading well.

Lanes not receiving resuspended Triton X-100 pellet sample or BlueRanger protein mixture (e.g., perhaps lanes 1 and 10) receive a corresponding amount of sample loading buffer. In the presently described use of the Bio-Rad mini-PROTEAN II system, both the chamber of the buffer tank (anode buffer) and the chamber of the electrode assembly/clamp assembly unit (cathode buffer) are filled up to a level a few mm above the upper edge of the small glass gel plate. So, the gel is submerged in running buffer and heat does not build up during electrophoresis.

The electrophoresis power supply is set at constant 200 V. At the beginning of electrophoresis, conditions are 97-98 mA and 19-20 W. In this procedure, what is defined as the tracking dye front is visually apparent below the position of the 220 kDa myosin band in lanes having BlueRanger protein standards. When the tracking dye front is about 6-7 mm above the bottom edge of clamp assembly (with the Blue Ranger myosin band about 6-7 mm above the resolving gel/plug gel interface), electrophoresis is terminated. Gel running times are 27-36 minutes. After electrophoresis, each 10% polyacrylamide plug gel band is discarded.

Gel fixation. Remove the gel and its two glass plates from their clamp assembly and place them onto a larger glass plate with the small glass plate of the electrophoresis gel on top. Use a metal spatula to laterally push away the 1.5 mm gray plastic spacer bars at either side of the gel and then to carefully pry up the small glass plate. Then use the end of the metal spatula to slice off most of each gel well divider and the upper corner of the gel at lane 1. Use a razor blade to remove the 5 mm 10% polyacrylamide gel.

Then place the gel/large glass plate into about 150 ml deionized water in an 8×8″ glass tray, rotate the glass tray horizontally clockwise and counterclockwise a little, and the gel will come off the large glass plate. Then remove the large glass plate from the glass tray. Composite agarose-polyacrylamide gels to be used for trypsin proteolysis and analysis by peptide mass spectrometry should not be fixed. Each gel intended for such use should be appropriately sliced with a razor blade or scalpel, with gel segments of interest then stored at −80° C. Likewise, gels intended for conventional Western blot electro-transfer of proteins onto nitrocellulose membrane or polyvinylidene difluoride membrane should not be fixed.

Gels intended for in-gel Western blot analysis (see below, DETAILED DESCRIPTION OF THE INVENTION, Section 2) or protein staining should be fixed by treatment with MeOH:glacial acetic acid:water (45:10:45 v/v/v). First, slide the gel into 100 ml fixer in an 8×8″ glass tray and put the tray on a platform rocker at 600 rpm for 30 minutes. Remove the fixer by aspiration, rinse gel momentarily with deionized water, and then repeat the fixation treatment for another 30 minutes. Then remove the second 100 ml of fixer, add 125 ml deionized water and put tray back onto a platform rocker for 30 minutes. Repeat the water rinse two more times. After this fixation procedure, the gel appears almost completely clear, with a tint of white color. The locations of the more prominent protein components are readily apparent by visual inspection as white bands or smears in the gel (as previously noted by Heinegard et al. 1985).

Staining of gels. In the presently described preferred embodiment of the invention, success in staining composite agarose-polyacrylamide gels has been achieved by default. The Perbio Science UnBlot™ In-Gel Chemiluminescent Detection Kit-Rabbit as sold cannot be used with CAPAGE gels. However, as described below in Section 2 of the DETAILED DESCRIPTION OF THE INVENTION, the present inventor has adapted this procedure so that it can be used to obtain in-gel Western blot images from such gels. Briefly stated, these new and novel changes include modification of (1) gel fixation; (2) gel handling; (3) gel rinsing; (4) the primary antibody incubation step; (5) the secondary antibody incubation step; (6) the chemiluminescent reagent; (7) film/gel exposure rack design and (8) the timing regimen for film exposure. These modifications are described below in detail in Section 2 of the DETAILED DESCRIPTION OF THE INVENTION. Subsequent to application of the modified in-gel Western blot procedure and gel rinsing with deionized water, gels are stored in PBS at 4° C. After several days of such storage, protein bands are found to have assumed a greenish-brown (GB) hue, while each gel otherwise is clear and colorless. This colorization process is referred to as GB staining. After the appearance of GB stained protein bands, each gel can be left in PBS or transferred into 0.01% NaN₃ for long term storage at 4° C. GB coloration of protein bands, a unique aspect of this invention, is stable for at least 18 months. For purposes of this disclosure, GB coloration of protein bands is defined as one form of optical density of stained composite agarose-polyacrylamide gels. For purposes of this disclosure, coloration of protein bands by use of commercially available gel electrophoresis protein stains well known to those of ordinary skill in the art are also defined as forms of optical density of composite agarose-polyacrylamide gels.

Digital processing of images. GB stained composite agarose-polyacrylamide gels were scanned at 2,000 pixels/inch with a UMAX Astra 2400S scanner/VistaScan Adobe Photoshop 7.0 system, and then digitally processed with Adobe Photoshop 6.0 or Adobe Photoshop CS3. Such gels were scanned in their intact wet state, not dried. The scanner lid was raised slightly so each gel was not compressed, and a sheet of aluminum foil (reflective side facing down) was placed over each gel so as to maximize the amount of reflected light recorded in each digital file.

Results. Protein molecular weight standards useful in work of this kind were not commercially available when the original inventive work described herein was done. To obtain an approximate idea of the molecular weight range covered in the presently described CAPAGE procedure, reference is made to the 0.5% agarose/2.0% polyacrylamide protein MW calibration curve of Perret et al. (1979). For purposes of discussing the presently described data, each gel lane is defined as being divided into ten 5.1 mm segments, segment 1 being the segment immediately below the loading well and segment 10 being at the bottom edge of the composite agarose-polyacrylamide gel. In the present procedure, Perbio Science Chemiluminescent BlueRanger protein molecular weight marker mix is pipetted into some loading wells of each gel. This product is intended for use in SDS/PAGE studies. It consists of a mixture of seven proteins, each covalently labeled with a blue dye. The largest protein in this mixture is myosin H peptide (MW 250 kDa). Once voltage was applied to a gel, electrophoresis was continued until the blue myosin H bands had run down to the lower part of gel segment 9. In doing so, the smaller proteins in the BlueRanger standard mix lanes were run into the 10% polyacrylamide plug gel or off the bottom edge of each gel.

Reference is then made to FIG. 4 of Perret and coworkers, comparing the location of the 250 kDa myosin H standard bands to the corresponding location of their 250 kDa human factor VIII-related protein monomer (referred to as DMS-1). Looking at FIG. 4 of Perret and coworkers, they described the full length of each gel lane as being 1.0 and the relative position of DMS-1 as being 0.725. For the presently described work, if the full length of a gel lane is defined as being 1.0, than the presently described myosin H bands run to a position of approximately 0.87. So, proteins in the presently described system run about (0.87/0.725) times further down each lane and therefore a correction factor of 1.2 is applied to convert the known MW data of Perret and coworkers to approximately corresponding positions on the presently described gels. Extrapolating the Perret and coworkers MW data to the presently described work, the approximate MW range of the 10 gel segment mid-points may be summarized as follows: segment 1 (from bottom of loading well), 30,000 kDa; segment 2, 18,000 kDa; segment 3, 9,500 kDa; segment 4, 4,500 kDa; segment 5, 2,700 kDa; segment 6, 1,225 kDa; segment 7, 750 kDa; segment 8, 500 kDa; segment 9, 255 kDa; and segment 10 (to bottom edge of resolving gel), 225 kDa.

Use of the presently described procedure is illustrated in FIG. S1 (not shown) of the biomedical journal article (text in preparation) corresponding to the instant disclosure. In each of the six panels the top (i.e., loading well) of each gel lane is to the left, and the bottom of the gel lane is to the right. Panel A illustrates the migration of the BlueRanger myosin H peptide to the lower part of gel segment 9. As noted in Perbio Science product literature, this standard protein sometimes migrates as two nearby bands. Panel B illustrates the gel lane profile corresponding to the sample of panel A, obtained by digital analysis using NIH ImageJ software. Panel C illustrates the GB staining image of a gel lane subsequent to electrophoresis of 364 μg of normal mouse spinal cord Triton X-100 precipitated protein. Panel D illustrates the gel lane profile corresponding to the sample of panel C. Panel E illustrates the GB staining image of a gel lane subsequent to electrophoresis of 406 μg of ALS mouse spinal cord Triton X-100 precipitated protein. Panel F illustrates the gel lane profile corresponding to the sample of panel E.

On the right side of panels C-F, a large protein band of ˜240 kDa is evident, and a smear of protein extends away from both the upper and lower sides of this band. As documented in the matrix-assisted laser desorption/ionization/time-of-flight mass spectrometry (MALDI/TOF MS) analysis of gel segments ALS-9 and CON-9 (see below, Sections 3-5 of the DETAILED DESCRIPTION OF THE INVENTION), this band contains evidence of numerous tryptic peptides characteristic of axonal cytoskeleton proteins (FIGS. S12 and S22, not shown, of the biomedical journal article [text in preparation] corresponding to the instant disclosure). Rather than being one protein, this band appears to represent a heterogeneous mixture of trypsinized remnants of the axonal cytoskeleton network.

Digitally magnified views of the gel segment 1-2 region of panels E and G of FIG. 1 (data not shown) of the biomedical journal article (text in preparation) corresponding to the instant disclosure have been illustrated in FIG. 2 (data not shown) of the biomedical journal article (text in preparation) corresponding to the instant disclosure. The arrows denote the distinct band of protein present at the bottom of each sample loading well. Although some of this material might be nerve cell cytoskeleton protein, much of it may well be extracellular matrix protein (e.g., collagen, tenascin and elastin). A thin film of protein is apparent at the bottom of each loading well, then a clear ˜1 mm gel zone, and then the band of protein aggregate ˜30,000 kDa in size. The ˜30,000 kDa protein aggregate region is below and separate from the protein that remained in the loading well. It is evident from this image that the physical texture of the ˜30,000 kDa protein aggregate band is not uniform, which suggests that its composition may be complex and heterogeneous.

2. Discussion. This invention disclosure section defines an electrophoretic procedure capable of isolating ultra high MW protein aggregates from nerve tissue. This work demonstrates that such protein aggregates are characteristic of normal murine spinal cord tissue. In the presently described procedure, Triton X-100 precipitation is used to obtain a protein fraction that is subsequently resuspended and subjected to resolution by gel electrophoresis. Triton X-100 treatment is a well-recognized procedure for precipitating cytoskeleton proteins. In the biomedical journal article (text in preparation) corresponding to the invention of the instant disclosure, evidence is presented supporting the preliminary conclusion that the ultra high MW protein aggregates described in the instant disclosure are indeed rich in tryptic peptides characteristic of axonal cytoskeleton proteins. Some modifications of the presently disclosed preferred embodiment of the procedure can be envisioned. For example, Triton X-100 precipitated protein samples can be sonicated by use of a micro-tipped probe sonicator, instead of a bath sonicator. Also, less AMP and less TEMED can be used to initiate polyacrylamide polymerization immediately prior to mixing with heated 1.0% agarose stock solution. This would allow for more time to pour each composite agarose-polyacrylamide gel.

The issue of well comb removal from newly made CAPAGE gels is a recognized problem. Well comb removal from SDS/PAGE gels is a simple process, as such gels do not adhere to the combs. But for CAPAGE gels, the polymer matrix does indeed stick to the well comb. If one attempts to remove a CAPAGE well comb, as one would do for an SDS/PAGE gel, the gel barriers between wells (i.e., “gel fingers” or “gel walls”) will be torn off with the comb. Also, the entire upper section of the CAPAGE gel will tend to be dislodged. Previous investigators have devised at least two ways of circumventing this problem. Some publications mention that gels were poured so that the gel barriers between wells were only about half of their usual height (Heinegard et al., 1985; Heimer and Sampson, 1987; Varelas et al., 1991). But while doing so facilitates well comb removal, it also limits the amount of sample volume that can be added to each well. Alternatively, Suh et al., (2005) have described a “triple comb” method for addressing this problem. In the casting of each 1.5 mm thick native CAPAGE gel, they used three 0.5 mm well combs. The middle comb can be removed first, and then the other two combs can be removed without damaging the gel barriers between wells. However, the Suh et al. (2005) well comb procedure requires precise positioning of the three well combs at the point of pouring a gel, complicating a method step that must be done quickly. The Suh et al. (2005) well comb procedure also predisposes for a degree of non-uniformity within each gel loading well, if the three well combs were not precisely aligned when inserted into position. The novel procedure for well comb removal as described herein was not anticipated by Suh et al. (2005) and has not been previously disclosed.

Previous studies have employed several different stains for visualizing proteins on CAPAGE gels. Such methods include the use of Coomassie Brilliant Blue R-250 (Counts et al., 1978; Gabelli et al., 1986; Maeda et al., 1991), use of Coomassie Bradford reagent (Suh et al., 2005) or use of toluidine blue stain (McDevitt and Muir, 1971; Varelas et al., 1991; Melrose et al., 2000; Knox et al., 2001). Conventional silver nitrate methods for staining polyacrylamide gels are not of use with agarose gels, as background staining is excessive. Still, the staining of dried agarose gels by use of silver nitrate in combination with tungstosilicic acid has been described (Lasne et al., 1983; Peats, 1984; Lubahn and Silverman, 1984). The Bio-Rad Silver Stain Plus Kit might work successfully on CAPAGE gels, but the work of the presently described disclosure did not explore this option. Lubahn and Silverman (1984) suggested that undried agarose gels are inherently unsuitable for silver staining, as hydrated agarose contains an excessive number of nucleation sites for silver deposition. So the applicability of silver staining for CAPAGE work is still somewhat unresolved. As the report of Steinberg et al. (2000) demonstrated that SYPRO Ruby IEF Protein Gel Stain (Molecular Probes) can efficiently stain proteins in either agarose or polyacrylamide gels, there is reason to believe that this fluorescent stain can also be used with CAPAGE gels.

The chemical basis for the GB residual staining of CAPAGE gels in the presently described study remains a riddle. But, taking into account the work of Mottley et al. (1991), it seems reasonable to propose that the development of such color is dependent upon the remaining horseradish peroxidase (HRP) activity present in the secondary antibody/HRP conjugate. After use of the in-gel Western blot imaging procedure described below in Section 2 of the DETAILED DESCRIPTION OF THE INVENTION, it appears that some residual secondary antibody/HRP conjugate remains stuck to the gel. Mottley and coworkers used electron spin resonance spectra analysis to demonstrate that HRP can oxidize malondialdehyde (MDA) to generate three iminoxyl radicals. They reported that such oxidation occurs only under mildly acidic conditions (e.g., pH less than 6.7). But it seems reasonable to propose that, under gel storage conditions of the presently described study, HRP present in the residual secondary antibody/HRP conjugate reacts with MDA, other carbonyl-containing substances and/or fatty acyl hydroperoxide precursors to generate chromophoric derivatives covalently linked to protein aggregates. The NaCNBH₃ reduction step of spinal cord homogenate preparation should have reduced labile Schiff base imines to chemically stable secondary amines. But NaCNBH₃ does not reduce free aldehydes (see page 216 of Fenaille et al. 2003). Likewise, NaCNBH₃ treatment would not affect fatty acyl hydroperoxide precursors that may be present, and which may generate aldehydes upon gel storage.

The data described above raise the question, why should ultra high MW protein aggregates of ˜30,000 kDa be detected in nerve tissue derived from a normal mouse? Two possible explanations may be offered. One explanation is that the occurrence of covalently linked aldehyde-protein complexes in nerve tissue of normal animals has been previously documented in immunohistochemical studies (Hall et al. 1998, Pedersen et al., 1998; Wataya et al., 2002). The other explanation is that transglutaminase-2 is known to be present in normal nerve tissue, and so it may serve a normal physiochemical role of keeping axonal cytoskeleton proteins covalently cross-linked to a certain extent (Selkoe et al., 1982; Miller and Anderton, 1986; Grierson et al., 2001). Taking these earlier studies into account, it is not surprising that CAPAGE analysis of normal mouse cytoskeleton proteins will show some evidence of ultra high MW protein complex molecular species. It may be that this is a normal state of affairs, and that this manageable degree of protein cross-linking is pushed past some tipping point in the etiologies of neurodegenerative diseases characterized by overt protein aggregation.

Section 2 Laboratory Method for Obtaining In-Gel Western Blot Images from Agarose/Polyacrylamide Gels

Section 1 described use of CAPAGE gels to resolve proteins and protein aggregates in the range of from ˜200 kDa to ˜30,000 kDa. In-gel Western blotting is a procedure whereby antibodies are used to specifically identify small amounts of proteins resolved in electrophoresis gels. Another aspect of this invention as defined in this section describes a method for obtaining Western blot film images based on in-gel analysis of 0.5% agarose/2.0% polyacrylamide electrophoresis gels. The preferred embodiment of this method is disclosed as follows. Various modifications of this preferred embodiment can also be envisioned that still reside within the metes and bounds of this disclosure. Such modifications include the use of nerve tissue samples obtained from humans or other animals, use of alternative grades of agarose, use of alternative concentrations of agarose, use of alternative grades of polyacrylamide, use of alternative concentrations of polyacrylamide, use of alternative primary antibodies, use of alternative secondary antibodies, use of alternative buffers, x-ray film capture of CAPAGE gel Western blot images at different time intervals, use of an alternative digital scanner to capture x-ray film visual images of CAPAGE gels, and other minor variations in methodological details.

Relatively few methods exist for the study of protein aggregates greater than 1,000 kDa. This procedure is a modification of the method for the commercially available Perbio Science UnBlot™ In-Gel Chemiluminescent Detection Kit-Rabbit, which is not designed or marketed for use in the study of protein aggregates greater than 1,000 kDa. The method presently described includes the use of a polyclonal primary antibody that is specific for the reduced form of the malondialdehyde/ε-amino-lysine Schiff base, an indicator of protein modification under conditions of oxidative stress. With use of this primary antibody, the present section describes the detection of oxidatively modified ultra high MW protein aggregates in normal adult mouse spinal cord tissue and transgenic B6SJL-TgN(SOD1-G93A)1Gur adult mouse spinal cord tissue. This procedure can correspondingly be used to study the ultra high MW protein aggregates that are characteristic of several neurodegenerative diseases.

Materials. An UnBlot™ In-Gel Chemiluminescent Detection Kit-Rabbit (catalog no. 33500), ImmunoPure Goat Anti-Rabbit IgG (H+ L) Peroxidase Conjugated (catalog no. 31460), and 5×7″ CL-Xposure film (catalog no. 34092) were from Perbio Science. Anti-malondialdehyde/ε-amino-lysine polyclonal antibody rabbit serum (catalog no. MDA 11-S) was obtained from Alpha Diagnostic International. Phosphate buffered saline tablets (catalog no. P-4417) were from Sigma-Aldrich.

Animals. A colony of transgenic B6SJL-TgN(SOD1-G93A)1Gur mice (Jackson Laboratories) and their non-transgenic littermate controls was maintained in the laboratory of Dr. Terry D. Heiman-Patterson at Drexel University College of Medicine. For the work illustrated in. FIGS. 1-3 (data not shown) of the biomedical journal article (text in preparation) corresponding to the instant disclosure and further described in Sections 1 and 2 of the DETAILED DESCRIPTION OF THE INVENTION, mice were 148 days old at sacrifice, near the maximum longevity for transgenic mice of this kind.

CAPAGE gel fixation and gel staining. Details of the CAPAGE method used in this disclosure section are presented above in Section 1. Based on visual observation of the blue colored myosin H peptide (MW 250 kDa) protein standard in gel lanes loaded with Chemiluminescent BlueRanger™ Protein Molecular Weight Marker Mix, it was apparent that significant amounts of proteins near the bottom of 0.5% agarose/2.0° A) polyacrylamide gels diffuse out of such gels into aqueous media within 24 hours, if such gels are not fixed. Gels intended for in-gel Western blot analysis or protein staining should be fixed by treatment with methanol (MeOH):glacial acetic acid:water (45:10:45 v/v/v). First, slide the gel into 100 ml fixer in an 8×8″ glass tray and put on a horizontal platform rocker at 600 rpm for 30 minutes. Remove the fixer by aspiration, rinse gel momentarily with 125 ml deionized water, and then repeat the fixer treatment for another 30 minutes. Then remove the second 100 ml of fixer, add 125 ml deionized water and put tray back onto a platform rocker for 30 minutes. Repeat the water rinse two more times. Such gels are stained by default, as a result of applying the presently described in-gel Western blot procedure. Subsequent to application of the in-gel Western blot procedure and gel rinsing with deionized water, gels are stored in PBS at 4° C. After several days of such storage, protein bands are found to have taken on a green/brown color (i.e., abbreviated GB staining).

In-gel Western blot procedure. This procedure is a modified version of the standard protocol (Instructions no. 1275.1) for the Perbio Science UnBlot™ In-Gel Chemiluminescent Detection Kit-Rabbit. For modification of the Hands-Off™ Incubation Colander, one of the long sides is removed with a band saw. The rough edges are then first sanded with course sandpaper on a wood block, and then sanded with fine sandpaper. In the following procedure, corresponding step numbers of the Perbio Science UnBlotT™ minstructions (version 1275.1) are noted in brackets.

(1) [PS step 2] Add the modified Hands-Off™ Incubation Colander into the glass tray used for fixation and use either a perforated piece of previously developed x-ray film or a Teflon culinary spatula to carefully slide the gel into the colander.
(2) [PS step 3] Place the modified Hands-Off™ Incubation Colander into a colander tray and pretreat the gel by addition of 50 ml of 50% MeOH. Gently shake gel for 15 minutes.
(3) [PS step 4] Place a standard 200 μl micropipette tip on rubber tubing attached to a vacuum trap and aspirate 50% MeOH out of tray. Add 100 ml deionized water to tray and wash gel with gentle shaking for 15 minutes. The gel can be left at this step overnight at 4° C.
(4) [PS step 5] Preparation of primary antibody solution. A vial of Alpha Diagnostics International MDA-11S serum is taken out of a −80° C. freezer and thawed to room temperature. Prepare 40 ml primary antibody solution by combining 36 ml phosphate buffered saline-Surfact-Amps™-20 (PBS-T, see page 3 of Instructions no. 1275.1 for the Perbio Science UnBlot™ In-Gel Chemiluminescent Detection Kit-Rabbit), 4 ml UnBlot™ 10× Antibody Dilution Buffer and 80 μl MDA-11S rabbit serum (i.e., a 1:500 v/v dilution of the serum).
(5) [PS step 6] Incubation of primary antibody with gel. Remove the 100 ml deionized water from the colander tray by aspiration. Add the primary antibody solution to the gel in its colander/colander tray. Put the gel with primary antibody into a ziplock plastic bag and put on cold room Lab-Line horizontal rotator at “continuous” time and speed setting 2.5 (i.e., very slow gentle rotation). Leave as such overnight.
(6) [PS step 7] Removal of primary antibody solution. Lifting the gel in its modified Hands-Off™ Incubation Colander, aspirate away the primary antibody solution from the colander tray. Remove droplets of primary antibody from the modified Hands-Off™ Incubation Colander, taking care not to touch the gel with the vacuum line micropipette tip. Add the first 100 ml PBS-T rinse and put gel/tray unit back in cold room on Lab-Line rotator at speed 3.0 for 1 hour. Repeat this rinsing procedure two more times.
(7) [PS step 8] Preparation of secondary antibody solution. Add 2 ml deionized water and 2 ml glycerol to a vial of lyophilized ImmunoPure Goat Anti-Rabbit IgG (H+L), Peroxidase conjugated. This secondary antibody stock solution should be stored at −20° C. Combine 80 μl of goat anti-rabbit IgG-horseradish peroxidase (HRP) stock solution with 36 ml PBS-T and 4 ml of 10× Antibody Dilution buffer (i.e., a 1:1,000 dilution).
(8) [PS step 9] Aspirate off the third 100 ml PBS-T rinse from step (6). Then add the 40 ml of diluted secondary antibody to the gel in its colander/colander tray. Leave the gel with secondary antibody on a cold room Lab-Line rotator at speed setting 3.0 for 1 hour.
(9) [PS step 10] Removal of secondary antibody solution. Lifting the gel in its modified Hands-Off™ Incubation Colander, aspirate away the secondary antibody solution from the colander tray. Remove droplets of secondary antibody from the modified Hands-Off™ Incubation Colander, taking care not to touch the gel with the vacuum line micropipette tip. Add the first 100 ml PBS-T rinse and put gel/tray unit back in cold room on Lab-Line rotator at speed 3.0 for 1 hour. Repeat this rinsing procedure two more times.
(10) Three additional PBS rinses (i.e., without Surfact-Amps™-20). Add two Sigma-Aldrich phosphate buffered saline tablets to 400 ml deionized water, dissolve, and then put on ice in cold room. When cold, aspirate away the third PBS-T rinse of step (9) and then add 100 ml PBS to the gel/tray unit. Put gel/tray unit on a room temperature Lab-Line rotator at speed 3.0 for 20 minutes. Repeat this rinsing procedure two more times.
(11) [PS step 11] Preparation of UnBlot™. Substrate Working Solution. Mix 10 ml UnBlot™ Stable Peroxide and 10 ml UnBlot™ Luminol Enhancer to make 20 ml of Substrate Working Solution.
(12) [PS steps 12-14] Incubation of gel with Substrate Working Solution. This is done in a 14×14 cm plastic weighing dish. The gel in its modified Hands-Off™ Incubation Colander is lifted out of its third PBS rinse, the colander is placed in a tilted position so that its open side is facing down into an empty plastic weighing dish, and deionized water is applied to slide the gel off of the colander and into the weighing dish. Then water in the dish is carefully removed by aspiration using first a vacuum line and then a 200 μl micropipettor. The 20 ml of Substrate Working Solution is then added to the dish, and the dish is gently swirled by hand for 5 minutes.
(13) [PS step 15] Final gel rinsing with water. A total of 50 ml deionized water is used. Remove the Substrate Working Solution by aspiration, quickly add about 20 ml water into the weighing dish, swirl water for about 10 seconds, remove water by aspiration, add the remaining water to the dish, swirl water for about 10 seconds, and then remove water by aspiration.

(14) [PS step 16] Preparation of gel for exposure of film. A standard metal x-ray film exposure cassette cannot be used for obtaining Western blot film images. This is because the lid of such a cassette would press down on one edge (or all) of the wet gel, distorting the image captured on film and damaging the gel. An efficient procedure for exposing film sheets to a gel is summarized as follows. After step (13), slide gel onto the center of a 6×6″ glass plate. Remove excess water from around the gel. Place four Bio-Rad 1.5 mm plastic gel spacer bars (catalog no. 165-2933) around the gel, and then use pieces of adhesive tape to secure spacer bars to the glass plate. From the UnBlot™ kit, remove a single two-ply cellophane sheet (catalog no. 1824765) and use scissors to remove their bound edge and separate the two layers. Trim a sheet of cellophane so that it is about 2 mm larger than the gel on each side. Place the trimmed cellophane sheet on top of the gel, taking care not to trap air bubbles beneath the cellophane while doing so, and then use four pieces of adhesive tape to secure its edges onto the four 1.5 mm plastic spacer bars. As required during the course of film exposures, add deionized water around edges of gel to remoisten; wick off excess water with Kim Wipes®. Reseal the top cellophane layer so as to ensure that there are no air pockets on either side of the gel. In place of a metal x-ray film exposure cassette, a shallow cardboard tray is used (e.g., the cardboard lid from a box of Avery Laser Labels). Any such cardboard tray will suffice, a tray about 8×11″ with a raised rim about ¼-½″ high. Place the 6×6″ glass plate with gel into the cardboard tray so that it is positioned into one corner, and then apply adhesive tape to the edges of the glass plate to secure it in this position. Cut a piece of ordinary Manila folder paper into a sheet small enough to fit into the cardboard tray.

(15) [PS step 17] Obtaining film images. Disposable latex gloves should not be worn, as such gloves tend to generate static electricity images on the films. In this procedure, the chemiluminescent signal will last for hours. In fact, when first taken in a dark room the entire gel will be glowing. This apparently is caused by either a residual background adhesion of Substrate Working Solution to the composite agarose-acrylamide gel or similar adhesion of the secondary antibody. To obtain a dark room film exposure, first place the cardboard tray on a table and secure it with several pieces of adhesive tape. Then position the piece of Manila folder paper on top of the cellophane sheet, with the Manila folder paper likewise slid into the corner of the cardboard tray over the gel. Place a sheet of film on top of the sheet of Manila folder paper, with the film also slid into the corner of the cardboard tray over the gel. While holding the film in position over the gel with one hand, the sheet of Manila folder paper is quickly pulled away from the cardboard tray. This exposes the film to the gel. Films are typically exposed to a gel for only 2-5 seconds, then quickly pulled away and developed.

From the time that the Substrate Working Solution is rinsed off the gel, for about 1 hour thereafter film images will be overexposed by excess chemiluminescent signal. So, any developed films exposed during this time will not show useful Western blot images. But after the first hour, the background chemiluminescent signal begins to significantly diminish and the specific in-gel Western blot image becomes progressively more apparent. Starting from about 1 hour after removal of the Substrate Working Solution, films should be exposed at about 10 minute intervals. During the film exposure process, the gel will be pale yellow, with numerous tiny air bubbles (about 1 mm across) all over the gel under the cellophane sheet. Yet these tiny bubbles do not appear on the films. Useful in-gel Western blot images can be obtained in a time frame of about 1½-3½ hours after removal of the Substrate Working Solution. These films will always show a degree of background chemiluminescence. But such residual background has a practical value in that it defines the edges of the gel. Subsequent to obtaining a digital file of a Western blot film, most of the residual background can then be subtracted by routine image processing.

(16) Storage of gels subsequent to Western blot film exposure. After capturing Western blot images on film, the gel has a pale yellow hue, but is almost colorless. There is no evidence of GB staining of protein bands. Store the gel in 50 ml PBS in its colander/tray in a ziplock bag in a cold room. After about 4 days, GB stained protein bands have appeared. Each gel can then be left in PBS or transferred into 0.01% NaN₃ for long term storage at 4° C. GB coloration of protein bands is stable for at least 18 months.

Digital processing of images. Western blot films were scanned at 2,000 pixels/inch with an Epson Perfection 4990 Photo Scanner/Adobe Photoshop 7.0 software system. To scan each film, it was first inserted into the Epson Perfection Western blot film holder. Then this film holder was inserted into its slot on the underside on the scanner lid, and the lid was closed. On the computer monitor, the “Adobe Photoshop 7.0” icon was clicked on. Then the “File” icon (upper left) was clicked on, and on its dropdown list the “Import” icon was clicked on. On the “import” icon dropdown list, the “Epson Perfection 4990 Photo scanner” icon was clicked on. Then the “Epson Scan” pop-up panel appeared. On the “Epson Scan” panel, the “Image type” was set on “Positive,” and in the “Original” sub-panel section the “document Type” was set on “Film (with area guide)” and the “Film Type” was set on “Positive Film.” Then the “Preview” icon was clicked on, and a digital dotted-line boundary (i.e., margin) was delineated around the Western blot gel image. Scanning details were set at 16 Bit grayscale and scan at 2,000 dpi. With such settings in place, each Western blot film was scanned. Each digital image was saved as both a psd file and a TIFF file. Saved Western blot images were then digitally processed with Adobe Photoshop 6.0 or Adobe Photoshop CS3. For purposes of this disclosure, such Western blot images are defined as yet another form of optical density of composite agarose-polyacrylamide gels.

Results. CAPAGE was used to resolve Triton X-100 precipitated spinal cord high MW protein aggregates from a 148 day old transgenic B6SJL-TgN(SOD1-G93A)1Gur mouse and a non-transgenic control mouse, followed by in-gel Western blot film imaging using anti-malondialdehyde/keyhole limpet hemocyanin (MDA/KLH) primary antibody (anti-MDA Ab). The antibody detects the presence of MDA, a bifunctional lipid peroxidation product covalently linked to lysine sidechain ε-amino groups. Such linkages are known as Schiff bases. These covalent linkages are somewhat labile, i.e., reversible. However, as each spinal cord homogenate was reduced with 50 mM NaCNBH₃, any Schiff bases present were transformed into chemically stable products. Correspondingly, the conjugated MDA/KLH antigen-used to inoculate a rabbit to generate the anti-MDA Ab serum had also been previously reduced with NaCNBH₃. If MDA reacts with lysine sidechains on two nearby peptides, it forms a covalent cross-link between them. In biological systems, this can result in the formation of protein aggregates. Such protein aggregates may be large enough to be seen by light microscopy, using anti-MDA Ab in immunohistochemical procedures (Hall et al. 1998). The present work describes the use of anti-MDA Ab to detect such ultra high MW protein aggregates derived from nerve tissue subsequent to electrophoretic isolation.

Protein molecular weight standards useful in work of this kind are not commercially available. To estimate the approximate MW range covered in the CAPAGE procedure of the presently described study, reference was made to the 0.5% agarose/2.0% polyacrylamide protein MW calibration curve of Perret et al. (1979). For purposes of discussing the presently disclosed data, each gel lane is described as being divided into ten 5.1 mm segments, segment 1 being the segment immediately below the loading well and segment 10 being at the bottom edge of the composite agarose-polyacrylamide gel. In this electrophoresis procedure, Perbio Science Chemiluminescent BlueRanger™ protein molecular weight marker mix was applied to some loading wells of each gel. This product is intended for use in SDS/PAGE studies. It consists of a mixture of seven proteins, each covalently labeled with a blue dye. The largest protein in this mixture is myosin H peptide (MW 250 kDa). Once voltage was applied to a gel, electrophoresis was continued until the blue myosin H bands had run down to the lower part of gel segment 9. In doing so, the smaller proteins in the BlueRanger™ standard mix lanes were run into the 10% polyacrylamide plug gel or off the bottom edge of each gel.

Reference was then made to FIG. 4 of Perret and coworkers, comparing the location of the presently reported 250 kDa myosin H standard bands to the corresponding location of their 250 kDa human factor VIII-related protein monomer (which they referred to as DMS-1). Extrapolating their MW data to the presently described work, the approximate MW range of the presently reported 10 gel segment mid-points may be summarized as follows: segment 1 (from bottom of loading well), 30,000 kDa; segment 2, 18,000 kDa; segment 3, 9,500 kDa; segment 4, 4,500 kDa; segment 5, 2,700 kDa; segment 6, 1,225 kDa; segment 7, 750 kDa; segment 8, 500 kDa; segment 9, 255 kDa; and segment 10 (to bottom edge of resolving gel), 225 kDa.

In the presently disclosed invention, several new and novel modifications of the standard Perbio Science UnBlot™ kit procedure were defined so as to adapt its utility to the imaging of composite agarose-polyacrylamide gels. Then the following hypothesis was tested: if the gel is left in a dark room long enough, will the intense background chemiluminescence diminish before the pattern of specific antibody/antigen labeling diminishes? To explore this question, the presently described method was used to expose a series of 19 films. The first of these films was exposed 17 minutes after step (13). Then additional films were exposed at approximately 10 minute intervals over a period of approximately 3 hours. Film exposure times varied from 1 second to 5 seconds, with most films exposed for 2 seconds.

As expected, within minutes after step (13), the entire gel was emitting an obvious yellow glow. This excessive background light emission completely overexposed film number 1 (exposure time 5 seconds) taken at 17 minutes after step (13). This resulted in an image that was nothing more than a black rectangle with fuzzy edges (data not shown). However, the signal-to-background issue then proceeded to improve. Film 2 (exposure time 2 seconds), exposed at 29 minutes after step (13), shows some evidence of protein band Western blot labeling, just barely observable on a light box against a still dark background (data not shown). When viewed on a light box, film 4 (exposure time 2 seconds) taken at 49 minutes after step (13) shows the complete pattern of specific antibody binding. But here too, the background light emission remained strong (data not shown). The most definitive film in this series was number 9 (exposure time 1 second), taken 91 minutes after step (13). The last film in this series, number 19 (exposure time 2 seconds) taken at 189 minutes after step (13), still showed an image with good signal-to-background, but some of the antibody labeled protein bands having the highest amounts of antigen showed more evidence of photo-bleaching (i.e., loss of Western blot chemiluminescent signal), as compared to corresponding data on earlier films in this series. All of the films in this series showed at least some degree of non-specific background chemiluminescence. However, with increasing time after step (13), non-specific background chemiluminescence progressively diminished, while specific antibody/antigen signal persisted.

Representative data are illustrated in FIG. S2 (not shown) of the biomedical journal article (text in preparation) corresponding to the instant disclosure, which illustrates digitally processed in-gel Western blot images of the CAPAGE gel segment 1/2 region. Images shown are (A) film 4 (exposure time 2 seconds, taken at 49 minutes after step 13), (B) film 6 (exposure time 2 seconds, taken 69 minutes after step 13), (C) film 9 (exposure time 1 second, taken 91 minutes after step 13), (D) film 12 (exposure time 2 seconds, taken 119 minutes after step 13), and (E) film 19 (exposure time 2 seconds, taken at 189 minutes after step 13). Adobe Photoshop™ digital processing of these images is summarized as follows: film 4 (Grayscale to RGB Color, 16 bits/Channel to 8 bits/Channel, Levels Input from 255 to 37, Curves y-axis 0-12-100-100-100, Brightness to +25, and Contrast to +25), film 6 (Grayscale to RGB Color, 16 bits/Channel to 8 bits/Channel, Levels Input from 255 to 32, Curves y-axis 0-12-50-87-100, Brightness to +10, and Contrast to +10), film 9 (Grayscale to RGB Color, 16 bits/Channel to 8 bits/Channel, Levels Input from 255 to 60, Curves y-axis 0-25-75-100-100, no change to Brightness, and no change to Contrast), film 12 (Grayscale to RGB Color, 16 bits/Channel to 8 bits/Channel, Levels Input from 255 to 70, Curves y-axis 0-12-50-87-100, Brightness to +10, and Contrast to −40), film 19 (Grayscale to RGB Color, 16 bits/Channel to 8 bits/Channel, Levels Input from 255 to 150, Curves y-axis 0-12-62-100-100, no change to Brightness, and no change to Contrast).

The in-gel Western blot images of lanes 5 and 6 clearly illustrate the ˜30,000 kDa protein aggregate band, which has a characteristic “W” shape. This demonstrates that the in-gel Western blot antibody labeling seen at the top of the gel lanes is not derived solely from material in the loading wells. The conclusion that the ˜30,000 kDa material is indeed covalently cross-linked ultra high MW protein aggregate is supported by (1) the inclusion of dithiothreitol in the gel sample loading buffer, (2) use of an electrophoresis buffer containing SDS (which dissociates peptides not covalently bound), and (3) the use of anti-MDA Ab for the in-gel Western blot film imaging.

Representative digitally processed in-gel Western blot images of the CAPAGE gel segment 9/10 region are illustrated in FIG. S3 (not shown) of the biomedical journal article (text in preparation) corresponding to the instant disclosure. This figure illustrates data from the films shown in FIG. S2 (not shown), with Adobe Photoshop™ processing as defined for FIG. S2. The in-gel Western blot images of the gel segment 9/10 region show the presence of a distinct protein band in the region of ˜255 kDa and a faint protein band in the region of ˜225 kDa, both of which possess epitopes for the anti-MDA Ab. The mid-range region of the gel lanes shown in FIGS. S2 and S3, i.e., segments 3-8, did not show evidence of additional protein bands by either GB staining or in-gel Western blot imaging, although corresponding tryptic peptide mass spectrometry data did show evidence of minor mid-range protein bands (e.g. FIG. 4, not shown of the biomedical journal article [text in preparation] corresponding to the instant disclosure).

Discussion. The standard protocol for the UnBlot™ In-Gel Chemiluminescent Detection Kit-Rabbit does not work on 0.5° A) agarose/2.0% polyacrylamide gels. The kit procedure as described by Perbio Science is not applicable for electrophoresis gels having less than 5% polyacrylamide. In particular, the present inventor was informed by a Perbio Science representative that gel concentrations commonly found in SDS/PAGE stacking gels (e.g., 3.5% polyacrylamide) will retain the secondary antibody of the UnBlot™ kit (personal communication). In the method presently disclosed, the standard Perbio Science kit protocol has been modified in several respects not obvious to those of ordinary skill in the art. These new and novel modifications include: (1) fixation of the gel; (2) modified gel handling (i.e., removal of one side of the Hands-Off™ Incubation Colander for gel sliding); (3) modified conditions in the primary antibody incubation step (i.e., doubling the volume; increase of incubation time from 1 hour to overnight; and incubation at 4° C. instead of at room temperature); (4) modified conditions in the secondary antibody incubation step (i.e., dilution of stock Ab 1:1 v/v with glycerol; and Ab final dilution of 1:1,000 instead of 1:250); (5) modified gel rinsing (i.e., three 1 hour rinses with PBS-T after 2° Ab incubation instead of three 10 minute rinses; then 3 additional 20 minute PBS rinses); (6) incubation with 2×volume of chemiluminescent reagent (i.e., UnBlot™ Substrate Working Solution) in a plastic weighing dish instead of kit colander tray; (7) design of an appropriate film/gel exposure rack (i.e., a cardboard tray with glass plate instead of a metal x-ray film exposure cassette) and (8) definition of a required new timing regimen for film exposure (i.e., best film imaging approximately 1%-3% hours after incubation with chemiluminescent reagent). This method can be expected to have equal utility as a modification of the Perbio Science UnBlot™ In-Gel Chemiluminescent Detection Kit-Mouse.

The findings presented in this section are based on use of a polyclonal primary antibody that is specific for the reduced form of the malondialdehyde/ε-amino-lysine Schiff base, an indicator of protein modification under conditions of oxidative stress. The presently described method has been developed as part of a study of axonal cytoskeleton protein cross-linking in the G93A superoxide dismutase 1 transgenic murine model of amyotrophic lateral sclerosis. As described elsewhere (e.g., FIG. 3, not shown, of the biomedical journal article [text in preparation] corresponding to the instant disclosure), the instant inventor's use of the presently described method did indeed produce preliminary evidence of increased formation of ultra high MW protein aggregates in murine ALS spinal cord tissue. But the presently described study also found evidence of a relatively low level of such protein aggregate formation even in normal mice. This observation is not without precedent. Published immunohistochemical studies have repeatedly demonstrated that spinal cord proteins from normal mice or normal humans are covalently linked with significant amounts of lipid peroxidation-generated aldehydes such as malondialdehyde and 4-hydroxynonenal, even in youth (Ferrante et al. 1997, FIG. 6; Hall et al. 1997, FIG. 3-A; Hall et al. 1998, FIGS. 6 and 7; Pedersen et al. 1998, FIG. 2; Wataya et al. 2002). Wataya et al. (2002) have proposed that the KSP repeat sequences of neurofilament-H and neurofilament-M may have evolved to serve a normal function of sequestering potentially toxic aldehydes in a relatively harmless manner, thus protecting the overall survival of nerve axons. Neurons must have a certain innate capability to manage this phenomenon (perhaps via autophagy), but it appears that this capability may be overwhelmed in neurodegenerative diseases such as ALS, Parkinson's disease, Alzheimer's disease, Huntington's disease and Kennedy's disease.

Future use of the presently disclosed method with primary antibodies specific for axonal cytoskeleton proteins may permit definition of the extent to which such ultra high MW protein aggregates consist of axonal cytoskeleton components. For example, antibodies of interest in this respect include those for neurofilament-L, neurofilament-M, neurofilament-H, dynein, dynactin, kinesin, plectin, bullous pemphigoid antigen proteins (i.e., BPAG1n1, BPAG1n2 and BPAG1n3; and ACF7), α-internexin and peripherin. Such studies may be extended to corresponding work on transgenic or knockout models of neurodegenerative disorders, as initially done with a transgenic murine model of ALS in the instant disclosure. Such future work might include studies on models of Alzheimer's disease, Parkinson's disease, diabetic polyneuropathy, Huntington's disease, Kennedy's disease, Down's syndrome and Charcot-Marie-Tooth disease (hereditary motor and sensory neuropathy). Other experimental animal models of human diseases can also be studied using the presently disclosed method, such as models of diabetic cardiovascular pathology or diabetic kidney pathology. Corresponding human biopsy samples (e.g., autopsy brain samples or peripheral nerve biopsy samples) can also be studied with this CAPAGE in-gel Western blot method. The extent to which antibodies raised against proteins in their native states will recognize their respective epitopes in oxidized protein aggregates remains to be determined. In this regard, the use of anti-MDA/ε-lysine polyclonal Ab or the use of other primary antibodies that are specific for epitope markers of oxidative stress (e.g., anti-4-hydroxynonenal/ovine serum albumin Ab, catalog no. 320-1EA, Cayman Chemical, Ann Arbor, Mich.) are of interest.

Previous reports have documented the use of CAPAGE, followed by conventional methods of electro-transfer and Western blotting (Heinegard et al. 1985; Gabelli et al. 1986; Hayward et al. 1991; Maeda et al. 1991; Melrose et al. 2000; Knox et al. 2001). However, such previous reports have also documented that the larger the size of the protein aggregate, the lower its recovery from a CAPAGE gel onto a conventional Western blot membrane (Elkon et al. 1984; Melrose et al. 1998). This selective loss of ultra high MW protein aggregate material when CAPAGE is combined with conventional Western blotting is a shortcoming of note in the prior art if one intends to study protein aggregation in neurodegenerative diseases. For these topics, the ultra high MW protein aggregates to be found near the tops of gel lanes are the subjects of particular interest. As disclosed herein, an attempt has been made to see if it would be possible to obtain Western blot images of such ultra high MW entities while they remain in the electrophoresis gel, and some success has been achieved in this effort. Hence, a deficiency inherent in the prior art has been overcome.

One curious aspect of the presently described methodology is the occurrence of composite agarose-polyacrylamide gel autocatalytic staining after use of the modified UnBlot™ in-gel Western blot procedure. When a used gel is rinsed with water after taking film exposures, the gel is pale yellow and no protein bands are apparent. Yet during the course of storage in PBS at 4° C. for several days, most protein bands on such gels become visible. Their color can be described as an amorphous combination of green and brown, abbreviated GB staining. Once formed, this color is permanent.

The chemical basis for GB staining remains a riddle. Its definition may require significant effort and lies beyond the limits of the instant disclosure. But, in a general sense, one possible explanation seems reasonable. Of the reagents used in this in-gel Western blot procedure, only two are known to remain affixed to each gel. These are the primary antibody and the secondary antibody/HRP conjugate. Mottley and coworkers (1991) have reported electron spin resonance evidence that HRP oxidizes MDA to generate three iminoxyl radicals. They reported that such oxidation occurs only under mildly acidic conditions (e.g., pH 4.6) and that electron spin resonance signals of such radical formation could not be detected above pH 6.7. However, the connection between such carefully defined in vitro studies and corresponding phenomena occurring in the presence of biologically derived MDA-protein conjugates is probably only qualitative. So, the findings of Mottley and coworkers infer that on the used composite agarose-polyacrylamide gels of the instant disclosure HRP is in immediate proximity with MDA and other carbonyl-containing substances, and that HRP catalyzes one or more reactions that generate chromophoric derivatives covalently linked to protein aggregates. It should also be noted that Triton X-100 spontaneously generates its own peroxide derivatives, and these carbonyl-containing products are substrates for HRP (Miki and Orii 1985). So the presently described GB staining might result from HRP reacting with Triton X-100 peroxides that are bound to proteins in a gel. This GB staining phenomenon is novel, unanticipated and fortuitous, as it provides a colorimetric form of gel image preservation subsequent to in-gel Western blot analysis.

### Section 3 Tryptic Peptide Mass Spectrometry

Another aspect of this invention as defined in this section is the new and novel practical adaptation of prior art methods of tryptic peptide mass spectrometry to the study and characterization of nerve tissue ultra high molecular weight protein aggregates isolated and thereby purified by use of the CAPAGE methodology described above in Section 1. In variations on the use of trypsin that fall within the metes and bounds of this disclosure, the endopeptidase used can be selected from the closed group consisting of chymotrypsin (EC 3.4.21.1, sequencing grade from bovine pancreas, Roche Diagnostics, Indianapolis, Ind.), endoproteinase Arg-C (EC 3.4.22.8, sequencing grade from Clostridium histolyticum, Roche Diagnostics, Indianapolis, Ind.), carboxypeptidase Y (EC 3.4.16.1, sequencing grade from yeast, Roche Diagnostics, Indianapolis, Ind.) optionally in combination with carboxypeptidases A and B, endoproteinase Asp-N (EC 3.4.24.33, sequencing grade from Pseudomonas fragi mutant, Roche Diagnostics, Indianapolis, Ind.), endoproteinase Glu-C (EC 3.4.21.19, serine protease V8 from Staphylococcus aureus V8, Roche Diagnostics, Indianapolis, Ind.) and endoproteinase Lys-C (EC 3.4.21.50, sequencing grade from Lysobacter enzymogenes, Roche Diagnostics, Indianapolis, Ind.).

Animals. A colony of transgenic B6SJL-TgN(SOD1-G93A)1Gur mice (Jackson Laboratories) and their non-transgenic littermate controls was maintained in the laboratory of Dr. Terry D. Heiman-Patterson at Drexel University College of Medicine. For the work illustrated in FIGS. 4-6 (not shown) of the biomedical journal article (text in preparation) corresponding to the instant disclosure, further illustrated in FIGS. S4-S38 (not shown) of the supporting online material (SOM) of the biomedical journal article (text in preparation) corresponding to the instant disclosure and described below in Sections 4 and 5 of the DETAILED DESCRIPTION OF THE INVENTION, mice were 59 days old at sacrifice.
In-gel trypsin digest protocol. The loading well of the ALS lane received approximately 397 μg of protein, while the loading well of the control lane received approximately 360 μg of protein. After CAPAGE, one murine ALS spinal cord homogenate gel lane was sliced into 10 segments (each of 5.1 mm) and one control littermate spinal cord homogenate gel lane was sliced into 10 segments (each of 5.1 mm). Samples were then trypsinized and subjected to matrix-assisted laser desorption/ionization/time-of-flight mass spectrometry (MALDI/TOF MS) using an Applied Biosystems 4700 Proteomics Analyzer. In variations on this procedure that fall within the metes and bounds of this disclosure, the mass spectrometer can be a matrix-assisted laser desorption/ionization-time-of-flight/time-of-flight tandem mass spectrometer (e.g., an Applied Biosystems 4800 tandem mass spectrometer), a hybrid linear ion trap-Fourier transformion cyclotron resonance (FT-ICR) mass spectrometer (Thermo Electron; San Jose, Calif.), a quadrupole ion trap mass spectrometer (Thermo Electron; San Jose, Calif.), a linear ion trap mass spectrometer (e.g., Finnigan™ LTQTM ion trap mass spectrometer equipped with a nanoelectrospray ion source (Thermo Electron, San Jose, Calif.), an ETD-enabled hybrid linear ion trap-orbitrap mass spectrometer (Thermo Fisher Scientific, Bremen, Germany) or an electrospray ionization tandem mass spectrometer. Composite agarose-polyacrylamide gel samples were prepared for MALDI/TOF MS analysis by use of the following procedure.
(1) After termination of electrophoresis, the screws on the Bio-Rad Mini-PROTEAN II clamp assembly pieces were loosened and the gel/glass plate unit was removed. The gel/glass plate unit was placed onto an 8×8 inch glass plate with the smaller gel glass plate on top. A small metal spatula was used to slide away the 1.5 mm Bio-Rad gray plastic spacer bar from each side of the gel. Then the spatula was used to slowly raise the small glass plate off of the gel. The spatula was used to slice off most of each of the gel fingers between the loading wells. The 8×8 inch glass plate was placed on top of an accurately positioned gel lane slicing guide diagram. A razor held by a hemostat and a small metal spatula were used to separate off and remove the 5 mm 10% PAGE plug gel from the bottom edge of the 0.5% agarose/2.0% polyacrylamide gel. The process of slicing gel segments was made more convenient and accurate by the investigator using a clear plastic thin layer chromatography spotting guide to cover and protect the unused area of a gel. (e.g., TLC Spotting Guide, Analtech Inc. product number 25-00). A large kitchen knife was used to slice along the entire length of the right and left sides of each gel lane of interest. Pressing down vertically with a single motion prevented any movement of the gel while the sides of each lane are being sliced. Then the razor/hemostat was used to slice each gel lane into ten segments, each being 5.1 mm from top to bottom. The razor/hemostat was tilted so that the blade was horizontal and it was used together with a small metal spatula to pick up each gel slice and deposit it in a 1.5 ml microfuge vial. After gel lane slicing was completed, the microfuge vials were momentarily centrifuged to bring the gel slice pieces down to the bottom of their vials. The vials were then stored at −80° C.
(2) Details of the trypsin treatment method used can be found online at http://ms-facility.ucsf.edu/ingel.html, with modifications noted as follows.
At UCSF step (4), the presently described samples were incubated in 25 mM NH₄HCO₃/50% CH₃CN for 30 minutes instead of 10 minutes.
At UCSF step (6), the presently described samples were subjected to steps (4) and (5) twice, not once.
At UCSF step (7), the presently described samples were air dried at room temperature for 1 hour, not dried in a Speed Vac. The presently described gel samples were not subjected to optional steps 7(a) to 7(f) of the UCSF method, which accomplish reduction with dithiothreitol and alkylation with iodoacetamide. The presently described CAPAGE gel loading buffer was 25% glycerol (v/v) in 0.5 M Tris pH 6.8 with 100 mM DL-dithiothreitol. So the presently described samples were already reduced.
At UCSF step (8), each of the presently described samples received approximately three-times as much Promega trypsin I (porcine trypsin, Fisher Scientific catalog number V5111A). Each of the presently described samples received 720 ng in 20 μl 25 mM NH₄HCO₃, while according to the UCSF method each sample would receive 250 ng in 20 μl 25 mM NH₄HCO₃.
At UCSF step (9), the presently described gel samples were rehydrated on ice for 30 minutes, instead of 10 minutes.
At UCSF step (10), the presently described samples were incubated at 37° C. for 4 hours, the minimum time period noted in the UCSF method.
The presently described procedure for extraction of peptides is as follows.
(1) Each sample was briefly vortexed, then centrifuged in a microfuge briefly. Then a micropipettor was used to transfer the trypsin supernate solution (about 5 to 10 μl) to new tube.
(2) Fifty microliters of 1% trifluoroacetic acid/50% CH₃CN was added to each gel segment to extract the peptides from the gel.
(3) These gel segment samples were vortexed briefly, sonicated for 30 minutes, centrifuged in a microfuge briefly, and then each supernate was withdrawn with a micropipettor.
(4) Each peptide extract solution was combined with its respective trypsin solution.
(5) Combined trypsin/extracted peptide supernate samples were dried in a Speed Vac.
(6) Each dried combined trypsin/extracted peptide supernate sample was resuspended In 6 μl of 0.1% trifluoroacetic acid/5% CH₃CN, then mixed well.
(7) Then 1 μl of each resuspended sample was mixed with 2 μl Working CHCA Solution. Working CHCA Solution was prepared as follows. Fifty microliters of a saturated solution of α-cyano-4-hydroxycinnamic (CHCA) acid was combined with 50 μl 0.3% trifluoroacetic acid/50% CH₃CN. To prepare a saturated solution of CHCA acid, a small amount of CHCA was mixed into an aliquot of 0.3% trifluoroacetic acid/50% CH₃CN, adding enough CHCA so that not all of it dissolved. Solid CHCA was allowed to remain, settling to the bottom.
(8) Then 1 μl of each sample was spotted onto a MALDI/TOF target plate well and spotted samples were left to air dry.
(9) In another target plate well, 1 μl of CAL2 Standard Mix was spotted for MALDI/TOF recording calibration. CAL2 Standard Mix was prepared as follows. It consisted of three components. Component (1) was 50% CH₃CN and 0.3% trifluoroacetic acid. Component (2) was a saturated solution of CHCA acid in component (1), as noted above. Component (3) was the CAL2 Mix from an Applied Biosystems Sequazyme Peptide Mass Standards Kit (catalog number P2-3143-00). The CAL2 Standard Mix used in the presently described work consisted of 9 μl of component (1), 9 μl of component (2) and 2 μl of the CAL2 Mix from an Applied Biosystems Sequazyme Peptide Mass Standards Kit.
(10) After completion of sample and CAL2 Standard Mix loading into wells of a MALDI/TOF target plate, samples were analyzed in an Applied Biosystems 4700 Proteomics Analyzer.
Operating and recording conditions of the mass spectrometer. An Applied Biosystems 4700 Proteomics Analyzer was used to obtain MALDUTOF MS recordings of tryptic peptide samples. Sample recording conditions were as follows: CID gas type—atmospheric, CID gas pressure—medium, calibration type—none, update default calibration—disabled, calibration type—internal, acquisition method—reflector positive, and processing method—reflector default with trypsin peak.
Printout of original (i.e., “raw”) tryptic peptide MALDI/TOF data. Applied Biosystems 4700 Data Explorer™ software was used to analyze copies of the 20 MS data files described in this invention disclosure. Applied Biosystems 4700 tryptic peptide MS data cannot be directly read by use of Microsoft Windows or Macintosh software. Copies of this data can only be accessed by use of Applied Biosystems 4700 Data Explorer™ software, which operates on either the Microsoft Windows 2000 operating system or on the Microsoft Windows XP/Service Pack 3 operating system.

Applied Biosystems 4700 Data Explorer™ software is of read-only format. Hence, the original raw data cannot be altered. However, by use of the Data Explorer™ software, minor changes can be made in the display of data. For example, several Data Explorer™ settings affect the appearance of the baseline of a spectrum trace file. Listed below are the exact steps taken to use the Data Explorer™ software to display each of mass spectrometry data files of this presently disclosed project.

(1) The CD containing the Data Explorer™ software was inserted into the computer CD drive and the software was opened. Then the CD was removed.
(2) The CD containing the raw Applied Biosystems 4700 data files was inserted into the CD drive.
(3) The inventor clicked on the “File” icon (upper left on monitor).
(4) The inventor clicked on “Open.” This displayed a panel that says “Select File(s)” on top and listed the nineteen raw data files for project:
(5) The inventor clicked on a raw data file to highlight it.
(6) The inventor clicked on the “Add” icon.
(7) The inventor clicked on the “Finish” icon.
(8) Then a panel appeared that said “Data Explorer. The data file (as selected) is read only . . . .” The inventor clicked on “Yes.” The raw data tryptic peptide spectrum trace (i.e., the total ion scan plot) then appeared in the larger upper monitor panel.
(9) On the bottom of the monitor screen, the inventor clicked on “Spec Peak List.” In the smaller lower monitor panel, the “Index List” of all individual recorded tryptic peptide ion masses for this sample appeared, with only the first two lines visible on the monitor.
(10) On the top line of the monitor screen, the inventor clicked on “Peaks,” then on its drop-down list click on “Peak Detection.” A panel then appeared that said “SPL Peak Detect Setup” on its top line. The inventor left “Integration Baseline Settings/(•) Valley to Baseline ( ) Valley to Valley” as is. In the “Peak Detection Settings” section, the inventor changed “% Centroid” from 50 to 1, changed “S/N Threshold” from 3 to 1, left “Noise Window Width (m/z)” on 250, left “(√) Recalculate S/N from Cluster Area” as is, changed “Threshold After S/N Recalculation” from 10 to 1, and then clicked on “OK.”
(11) On the top of the monitor screen, the inventor clicked on “Process.” On the drop-down list, the inventor clicked on “Advanced Baseline Correction.” the inventor changed “Peak Width” from 32 to 2, left “Flexibility” as is at 0.5, left “Degree” as is on 0.1, and then clicked on “OK.”
(12) On the top line of the monitor screen, the inventor clicked on “Peaks.” On the drop-down list, the inventor clicked on “Filter Peak List.” For the check box to the left of “Enable Peak List Filter,” the inventor deleted the check. The inventor left the check in the box for “Nonisotopic,” and for “Charge State” left its check box blank and left its value at zero. Then the inventor clicked on “OK.”
(13) Then the inventor clicked on the “File” icon on the top line of the monitor screen, clicked on “Print,” and clicked on “Print Spectrum Trace.” On the printer panel, the inventor selected the desired printer. For this work, an HP LaserJet 4000 Series PCL printer was used.
(14) The inventor clicked anywhere on the smaller lower panel on the monitor screen, then clicked on the “Print” option on the pop-up list. Then the printer panel appeared. The printer to be used was selected, then the inventor clicked on “OK.”
(15) After the complete line item list of all tryptic peptide ion masses of the sample was printed (typically, from about 2,000 to about 4,000 lines of data), the inventor clicked on the “X” Exit icon on the upper right of the monitor screen. A panel appeared that said “Processing History (sample name). Do you want to save the file's processing history?” The inventor clicked on “Save.”
It should be noted that, although the Applied Biosystems 4700 Data Explorer™ software operates on either the Microsoft Windows 2000 operating system or on the Microsoft Windows XP/Service Pack 3 operating system, it is not possible to directly store a data file in Microsoft Windows. Hence, for each of the tryptic peptide MALDI/TOF MS spectrum traces (i.e., MS total ion plots, data not shown), it was necessary to print the spectrum trace, then digitally scan it and save the image as a JPEG file (i.e., FIGS. S4-S23 of the biomedical journal article [text in preparation] corresponding to the instant disclosure). Each of the Applied Biosystems 4700 Data Explorer™ line-by-line tryptic peptide files (i.e., its Applied Biosystems 4700 Spectrum Peak List) was printed, and these printouts were manually inspected by the present inventor to obtain the results disclosed herein. No software presently exists to perform such analysis.
UniProtKB/Swiss-Prot or UniProtKB/TrEMBL tryptic peptide reference files used as prior art reference information in the instant disclosure. With the exception of human (Cu,Zn)-superoxide dismutase (file P00441), all other files represent murine proteins. The list of reference files includes:
alsin (Swiss-Prot file Q920R0)
amyloid beta A4 protein (β-amyloid peptide 1-40, Swiss-Prot file P12023)
amyloid beta A4 protein (β-amyloid peptide 1-42, Swiss-Prot file P12023)
androgen receptor (Swiss-Prot file P19091)
brain-derived neurotrophic factor (Swiss-Prot file P21237)
bullous pemphigoid antigen 1 (dystonin, Swiss-Prot file Q91ZU6 isoforms 1/2/3/4)
cadherin-2 (neural cadherin, Swiss-Prot file P15116)
caspase-3 (Swiss-Prot file P70677)
cytochrome c oxidase polypeptide VlIc (Swiss-Prot file P17665)
cytoplasmic dynein 1 heavy chain 1 (dynein heavy chain, cytosolic, Swiss-Prot file Q9JHU4)
dystrophin (Swiss-Prot file P11531)
E3 ubiquitin-protein ligase HECW1 (Swiss-Prot file Q8K4P8)
E3 ubiquitin-protein ligase HUWE1 (Swiss-Prot file Q7TMY8)
E3 ubiquitin-protein ligase parkin isoform 1 (Park2, Swiss-Prot file Q9WVS6)
E3 ubiquitin-protein ligase RNF19A (dorfin, Swiss-Prot file P50636)
E3 ubiquitin-protein ligase RNF216 (Swiss-Prot file P58283)
granulin-epithelin precursor (Swiss-Prot file P28798)
hamartin (Swiss-Prot Q9EP53 isoform 1)
huntingtin (Swiss-Prot file P42859)
huntingtin-associated protein-1 (Swiss-Prot file 035668)
kinesin heavy chain isoform 5C (Kif5c, Swiss-Prot file P28738)
kinesin-like protein (KIF1A, Swiss-Prot file P33173)
MICAL-2 (Swiss-Prot file Q8BML1)
microtubule-associated protein tau (Swiss-Prot file P10637)
neurofilament-66 (Swiss-Prot file P46660)
neurofilament heavy polypeptide (neurofilament-H, Swiss-Prot file P19246)
neurofilament-L (Swiss-Prot file P08551)
neurofilament-M (Swiss-Prot file P08553)
neuronal kinesin heavy chain (NKHC, Swiss-Prot file P33175)
peripherin (Swiss-Prot file P15331)
porcine trypsin (Swiss-Prot file P00761)
presenilin 1 (Swiss-Prot file P49769)
presenilin 2 (Swiss-Prot file Q61144)
probable helicase senataxin (Als4 protein homolog, Swiss-Prot file A2AKX3 isoform 1)
synaptojanin 2 (Swiss-Prot file Q9D2G5)
TAR DNA-binding protein 43 (Swiss-Prot file Q921F2)
transitional endoplasmic reticulum ATPase (valosin-containing protein, Swiss-Prot file Q01853)
ubiquilin-1 (Swiss-Prot file Q8R317)
ubiquilin-2 (Swiss-Prot file Q9QZMO)
ubiquilin-4 (Swiss-Prot file Q99NB8)
ubiquitin (Swiss-Prot file P62991)
ubiquitin-activating enzyme E1-1 (Swiss-Prot file Q02053)
ubiquitin-conjugating enzyme E2 K (Swiss-Prot file P61087)
ubiquitin-conjugating enzyme E2 E2 (Swiss-Prot file Q91W82)
ubiquitin-conjugating enzyme E2 E3 (Swiss-Prot file P52483)
ubiquitin conjugation factor E4 B (Swiss-Prot file Q9ES00)

### Generation of Tryptic Peptide Gel Lane Profile Graphs by Analysis of Mass Spectrometry Data.

For each of the 20 gel segment samples, both a tryptic peptide MALDI/TOF MS spectrum trace for the ion mass range of 800-4,000 Daltons (i.e., a tryptic peptide MS total ion plot, FIGS. S4-S23 of the biomedical journal article [text in preparation] corresponding to the instant disclosure) and a table of the tryptic peptide ion mass data were printed.

In each tryptic peptide MS total ion plot (data not shown, i.e., FIGS. S4-S23 of the biomedical journal article [text in preparation] corresponding to the instant disclosure), the red circle denotes the trypsin auto-catalytic peptide ion mass at 842.5094 Da, used as the internal standard in this work. For brevity, once stated to the fourth decimal point, each tryptic peptide ion mass is stated as it's nearest whole number. The largest ion mass peak in any sample is referred to as the “base peak,” arbitrarily set at 100%. The “relative intensity” of any other ion mass peak in the sample is its percentage relative to the base peak. The trypsin 842 ion mass peak is the base peak in 17 of the 20 samples, the exceptions being ALS-1, ALS-9 and CON-10. In order to statistically correct for the fact that trypsin 842 was not the base peak in three gel segment samples, ion mass relative intensity data in ALS-1 was multiplied by 1.47(data not shown), ion mass relative intensity data in ALS-9 was multiplied by 3.48 (data not shown), and ion mass relative intensity data in CON-10 was multiplied by 1.13 (data not shown). In doing so, all data represented in the 20 gel samples is numerically compared to a relative ion mass peak intensity of trypsin 842 set at 100%.

With use of the Data Explorer™ display settings as defined above in Section 3, sub-section Printout of original (i.e., “raw”) tryptic peptide MALDI/TOF data, the MS total ion plot for gel segment CON-6 showed a baseline shifted up the left-side y-axis (i.e., percent intensity relative to base peak trypsin ion mass 842 Da) from zero to 9.57 (FIG. S19 of the biomedical journal article [text in preparation] corresponding to the instant disclosure, not shown). This display artifact is due to the extremely small total amount of material in MS sample CON-6. This is documented by the value of 10.1 at the top of the right-side y-axis, a value which is substantially lower than that of any of the other 19 samples. To correct for this single sample baseline shift artifact, CON-6 tryptic peptide ion mass relative intensity values used in plotting the inventor's gel lane profiles were corrected by subtraction of 9.57.

The reader's attention is drawn to the fact in ALS-1 there are five ion mass peaks larger than trypsin 842 (FIG. S4 of the biomedical journal article [text in preparation] corresponding to the instant disclosure, not shown). These are located at 836, 981 (the base peak), 1455, 1732 and 2160. Extensive Swiss-Prot MS database searching has provided reasonable, yet provisional, identifications of these five unusually large ion mass peaks. As the presently disclosed tryptic peptide MS data constitutes only an introductory proof-of-concept disclosure, all protein identifications and conclusions are provisional, and are intended to illustrate the utility of the instant new invention.

The tryptic peptide MS total ion plots for gel segment ALS-1 (FIG. S4, not shown) and gel segment CON-1 (FIG. S14, not shown) indicate extremely complex patterns of peptide data, but they also suggest—even by visual inspection—that there is more protein in the ALS sample. These samples represent proteins or, more accurately, protein complexes in the range of about 30,000 kDa. With reference to the Swiss-Prot mouse protein tryptic peptide database, the present inventor has provisionally identified several axonal cytoskeleton proteins and other proteins of interest that are concentrated in gel segment ALS-1, using this tryptic peptide MS file as a “Rosetta stone” for the decipherment of ALS axonal protein aggregate content.

The 20 raw MALDI/TOF MS data files have a total of 133,952 lines of tryptic peptide ion mass data. Unfortunately, there is no online software designed to identify such extremely complex mixtures of peptides as those represented in this data. The “Aldente” Swiss-Prot software has proven useful for generating lists of candidate proteins that might explain the larger ion mass peaks. But, after typing in one or more tryptic ion masses, Aldente generates a list of candidate proteins that may exceed more than 100 Swiss-Prot tryptic peptide MS files, and these must then be examined one-by-one. In future studies of this kind, it would be preferable to switch from MALDI/TOF MS analysis to tandem MS, which is more definitive and also lends itself to automated data analysis by presently existing software.

The present inventor printed out Swiss-Prot tryptic peptide MS data files for proteins of interest one at a time, and then compared each list to the contents of the ALS-1 MS tryptic peptide file. In general, only ion masses having an uncorrected relative intensity >20 and peak area >20 in the MS raw data printout of gel segment ALS-1 have been considered within the instant disclosure as discussed below and in the biomedical journal article (text in preparation) corresponding to the instant disclosure. Such a “ 20/20” restriction limits consideration to those ion mass concentrations in the top eight percent of the 6908 lines of raw tryptic peptide data present in ALS-1. However, in some cases where curiosity warranted doing so, ion masses less than 20/20 relative intensity/peak area were analyzed by the present inventor. These include several ion masses “unique” for tau protein, caspase-3, presenilin 1, presenilin 2, cadherin-2 and transgenic human (Cu,Zn)-superoxide dismutase (G93A hSOD1).

Each detectable peptide is basically a loop of amino acids that extended off the surface of protein aggregate particles, making it accessible to attack by trypsin. The present inventor surmises that the ability of trypsin to cleave peptides off of covalently bound protein aggregates is only nominal, leaving much aggregate intact. Indeed, after completion of the trypsin treatment and peptide extraction procedure (see above), tiny pellets of residue were left in the original microfuge vials, with pellet sizes approximately corresponding to the amount of protein aggregate originally in each gel segment according to GB gel staining images and the tryptic peptide MALDI/TOF mass spectrometry spectrum traces (i.e., MS total ion mass plots) of gel lane segment samples. With trypsin use as presently defined, recovered ion mass signals were less than ideal. In future work, steps can be taken to increase trypsin efficacy and yield of peptides (e.g., see Park and Russel, 2001), such modifications being presently defined as falling within the metes and bounds of the instant disclosure.

In the inventor's figures of tryptic peptide gel lane profiles, peptide MS data representative of one protein or representative of a group of proteins having similar cellular functions have been illustrated (FIGS. 4-6 and SOM FIGS. S24-S38 of the biomedical journal article [text in preparation] corresponding to the instant disclosure, data not shown). These figures illustrate protein aggregate size over the MW range from approximately 225-30,000 kDa. In each of the tryptic peptide ion mass electrophoresis gel lane profiles so presented, the x-axis represents gel lane segments 1-10. In these figures, the y-axis represents ion mass relative intensity, as compared to the value of trypsin 842 as internal standard (arbitrarily set at 100%). Tryptic peptide ion masses represented in the inventor's figure legends are rounded off to the nearest whole number. With the briefly discussed exception of transgenic G93A human (Cu,Zn)-superoxide dismutase, all of the proteins discussed herein are genetically normal polypeptides of murine origin. In the instant disclosure, reference to a tryptic peptide as being “unique” means that in within the limits of the present inventor's review of Swiss-Prot tryptic peptide files, a particular tryptic peptide ion mass was found to be uniquely characteristic of only one parent protein among those studied and considered reasonably likely to be present in spinal cord tissue. However, some data has been developed and studied by the present inventor that represent two parent proteins of the same functional class (e.g. tryptic ion mass 981, characteristic of both neurofilament-M and neurofilament-66).

### Section 4 Normal Homologs of ALS-Related Proteins

Mutant forms of alsin (the product of the ALS2 gene) and senataxin (the product of the SETX gene) are known to cause ALS (Orban et al., 2007). In an introductory attempt to learn more about possible relationships between different forms of ALS, the presently described MS database was screened for evidence of peptide ion masses characteristic of the mouse homolog of normal alsin. The gel lane profiles for five alsin “unique” _{>}20/>20 ALS-1 tryptic ion masses are illustrated in FIG. S24 (not shown) (ion masses 952.5323, 1027.6258, 1028.4982, 1366.8052, and 1444.7543 Da). These gel lane profiles suggest that alsin is preferentially incorporated into the ultra high MW protein aggregate found to be enriched in ALS-1. For these tryptic peptide ion masses, the average ALS-1 relative intensity is about three-times as high as its CON-1 counterpart. This raises the possibility that in the G93A hSOD1 state, genetically normal alsin may be more likely to become entwined into ultra high MW protein aggregates and supports the proposal that alsin plays a role in vesicle transport or membrane trafficking processes (Orban et al., 2007).

Mutations in the human senataxin gene have been found to cause juvenile ALS (Chen et al., 2004). Murine senataxin is a relatively large protein (SEN, MW 297.6 kDa), but only one “unique” ALS-1 peptide at >20/>20 relative intensity/area was found (ion mass 2013.0368 Da, FIG. S25, not shown). Here CAPAGE gel segment ALS-1 has about twice as much of this peptide as found in gel segment CON-1. Although this is only a single ion mass, it is a large tryptic peptide (18 Amino acids). As such, the data plots for the 2013 Da tryptic peptide ion mass may be of some significance. Another twenty-six >20/>20 ALS-1 ion masses were also found for SEN that were additionally characteristic for at least one other protein investigated in this work.

Mutations in the human progranulin gene have been recently identified as the cause of some forms of frontotemporal dementia with ubiquitin-positive intraneuronal inclusion pathology [FTLD-U (Baker et al. 2006, Cruts et al. 2006)] and have been linked to some forms of ALS (Schymick et al. 2007, Sleegers et al. 2008). With this in mind, the present inventor also examined the MALDI/TOF MS tryptic peptide database to see if there is evidence suggesting increased incorporation of murine progranulin in the ALS ultra high MW aggregates. This protein is also known as granulin-epithelin precursor [Swiss-Prot file P28798 (Daniel et al. 2003)]. Only one “unique” tryptic peptide was found to have ion mass >20/>20 relative intensity/peak area (FIG. S26, not shown). This is Swiss-Prot peptide 1412.6337. Its relative intensity in the ALS-1 file printout is 35.12, while the corresponding value in the CON-1 gel segment was 12.02. It is characteristic of a peptide having 13 amino acids. As this tryptic peptide is fairly large, it is relatively likely that it's presence represents only one protein. So, the 1413 Da tryptic peptide ion mass data implies that an increased amount of progranulin may be present in of ALS-1 gel segment. But murine progranulin is a relatively small protein (63.5 kDa) and five other tryptic peptides having ion masses >20/>20 relative intensity/area were found to also be characteristic of other proteins searched in this study.

Mackenzie et al. (2007) reported increased levels of TAR DNA-binding protein 43 (TDP-43) in sporadic human ALS samples. But Mackenzie et al. also reported that samples from human cases of mutant SOD-1 ALS, analogous to the transgenic murine ALS model used in the presently reported study, do not show evidence of TDP-43 aggregation. With a molecular weight of 44.5 kDa, murine TDP-43 is a relatively small protein, having only 19 tryptic peptides. Only one Swiss-Prot tryptic peptide for murine TDP-43 was found to have an ion mass >20/>20 relative intensity/area in the present inventor's CON-1 gel segment sample and is apparently “unique” (FIG. S27, not shown). This is Swiss-Prot peptide 1144.5521 Da. Its relative intensity in the CON-1 file printout is 29.35 and its peak area is 39.4, while the Corresponding relative intensity and peak area values in the ALS-1 gel segment were zero. Furthermore, the control mouse gel lane has a secondary peptide peak for this tryptic peptide in the middle region of the lane. So, some evidence for provisional detection of TDP-43 exists in the presently disclosed tryptic peptide database, but predominantly in the control mouse spinal cord homogenate, not in the ALS spinal cord homogenate. These provisional observations appear to be in general agreement with the findings of Robertson et al. (2007), who reported no evidence of TDP-43 mislocalization from nuclei to cytoplasm in motor neurons of G93A hSOD1 transgenic mice and no association of TDP-43 with ubiquitinated inclusions in this model.

The role of mitochondria in ALS etiology has attracted some interest (Fujita et al., 1996; Liu et al., 2004), and defects in mitochondrial function in transgenic G93A hSOD1 mice have been reported (Kirkinezos et al., 2005). Mitochondrial dysfunction also appears to be involved in the etiologies of Alzheimer disease, Parkinson disease and Huntington disease (Perry et al., 2003; Rossi et al., 2004; Zhu et al., 2004). Cytochrome c oxidase mediates the final step of electron transfer reactions in the respiratory chain, catalyzing the transfer between cytochrome c and molecular oxygen and concomitantly pumping protons across the inner mitochondrial membrane. In the etiology of ALS, it appears that neuronal mitochondria are prone to rupture at an early stage in the course of the disease, releasing their contents. As this involves the release of enzymes involved in electron transport, the rupture of mitochondria has the effect of exacerbating oxidative stress within the immediate surroundings. Hence, mitochondrial dysfunction may play a role in the initiation and development of cytoskeleton protein cross-linking and aggregation in affected ALS neurons, and a similar course of events might be part of the etiologies of other neurodegenerative diseases.

In the presently described disclosure, the tryptic peptide MS database was screened for evidence of peptides that are members of the cytochrome c oxidase complex. The hypothesis being considered was that, once mitochondria had ruptured, cytochrome c oxidase peptides originally associated with the mitochondrial inner membrane might be exposed to cross-linking and aggregating cytoskeleton proteins, and might become incorporated into such protein aggregates. The only “unique” peptide present in ALS-1 at >20/>20 relative intensity/peak area that is characteristic of cytochrome c oxidase polypeptide VIIc (Cox7c) is ion mass 2338.2126 Da (FIG. S28, not shown). Cox7c is small, at 5.4 kDa, and its Swiss-Prot tryptic peptide list contains only three entries. For Cox7c, the ion mass at 2338 Da is the largest Swiss-Prot peptide, having 21 amino acids. Hence, it is highly indicative for the presence of Cox7c.

The data represented in FIG. S28 suggest that the cytochrome c oxidase peptide may have been incorporated into the ultra high MW protein aggregate characteristic of gel segment ALS-1. Many histological and immunohistochemical studies have shown evidence of ruptured mitochondria (sometimes described as “mitochondrial ghosts”) in the immediate proximity of ALS proximal axonal cytoskeleton protein aggregates. But, the presently disclosed work appears to take this cytological association one step further, implying that proteins normally found associated with mitochondrial inner membranes may actually be integrated into ALS ultra high MW protein aggregates.

Ubiquitin-conjugating E2 enzymes and related proteins are illustrated in the panels on the right side of FIG. 5 (not shown) of the biomedical journal article (text in preparation) corresponding to the instant disclosure. Comparable data for tryptic peptides characteristic of ubiquitin-protein E3 ligases are illustrated in FIG. S29 (not shown). In FIG. S29, protein name abbreviations are: E3 ubiquitin-protein ligase HUWE1, HUWE1; E3 ubiquitin-protein ligase HECW1, Q8K4P8; and E3 ubiquitin-protein ligase RNF216, RNF216. The tryptic ion masses shown are: 1050.5136 Da (HUWE1/Q8K4P8), 1134.4666 Da (RNF216), 1167.5065 Da (HUWE1), 1464.7436 Da (HUWE1), 1555.7573 Da (Q8K4P8), 1586.8206 Da (HUWE1), 1601.8017 Da (HUWE1), and 1743.8020 Da (RNF216).

The ubiquilins (FIG. S30, not shown) are a class of proteins that interact with cytoskeleton proteins and contain ubiquitin domains. In FIG. S30, protein name abbreviations are: ubiquilin-1, UBQL1; ubiquilin-2, UBQL2; and ubiquilin-4, UBQL4. The tryptic ion masses shown are: 1430.8001 (UBQL4), 1762.8904 (UBQL2), 1857.8591 (UBQL4), and 1909.9589 (UBQL1). As shown in FIG. S30, the “unique” tryptic peptide ion masses characteristic of UBQL1, UBQL2 and UBQL4 in gel segment ALS-1 are, on average, about four- to five-times higher than the corresponding values in gel segment CON-1. As ubiquilins interact with presenilins, they may play a role in Alzheimer's disease (AD) etiology (Mah et al., 2000). Immunohistochemical studies on human brain samples have shown that anti-ubiquilin antibodies robustly stain neurofibrillary tangles in AD samples and Lewy bodies in Parkinson disease samples (Mah et al., 2000). Bertram et al. (2005) have reported that a genetic variant in the gene for ubiquilin 1 is associated with an increased risk of AD. Also, ubiquilin 1 and ubiquilin 2 have been shown to be components of neuronal aggregates in a murine model of Huntington disease (Doi et al., 2004).

Section 5 Preliminary MALDI/TOF MS Data Regarding Concomitant Aggregation in the Transgenic G93A hSOD1 Murine Amyotrophic Lateral Sclerosis Model of Proteins Commonly Associated with Other Neurodegenerative Diseases

With the in vitro protein aggregate isolation and peptide-MS procedure described in the present disclosure, the inventor has been able to (1) search for the presence of a wide range of cytoskeleton proteins and proteins related thereto, (2) obtain a profile of protein incorporation into aggregates over the size range of from ˜225 to ˜30,000 kDa, and (3) qualitatively estimate the amount of each protein component in aggregates of various sizes. There is reason to believe the state of oxidative stress characteristic of the transgenic G93A hSOD1 murine model of ALS may also affect axonal proteins recognized as metabolic markers for other neurodegenerative diseases. In another aspect of this invention as defined in this section, the present inventor has defined preliminary data suggesting that the presently described methodology can detect and monitor the incorporation of such protein metabolic markers into ultra high MW protein aggregates.

The expanded polyglutamine forms of huntingtin (HD) and androgen receptor (ANDR) have been implicated in the disruption of normal axonal transport (Szbenyi et al., 2003; Gunawardena et al., 2003). Apparently, the normal forms of these proteins play roles in axonal transport. So, in the presently described disclosure it was of interest to see if tryptic peptides characteristic of either of the normal mouse homologs of these proteins might be incorporated into the ultra high MW protein aggregate found predominantly in gel segment ALS-1.

The gel lane profiles for six HD “unique”>20/>20 ALS-1 tryptic peptide ion masses are illustrated in FIG. S31 (not shown). The ion masses are 1130.5741, 1562.8285, 1701.9785, 2058.9648, 2127.1907, and 2160.1270 Da. Another twenty-three >20/>20 ALS-1 ion masses were also found for HD that were additionally characteristic for at least one other protein investigated in this work (data not shown). The MW of this protein is 344.7 kDa. The FIG. S31 data suggest that HD is preferentially incorporated into the ultra high MW protein aggregate found to be enriched in ALS-1. For five of the ion masses, the ALS-1 relative intensity is about twice as high as its CON-1 counterpart. However, for ion mass 2160 Da, the ALS-1 value is more than four-times higher than its CON-1 counterpart. This ion mass relative intensity in ALS-1 is another one of the five ion masses present at concentrations exceeding that of trypsin 842. The HD 2160 Da ion mass has 21 amino acids in it. Hence this is a very large tryptic peptide and not likely to have originated from some other parent protein. Furthermore, the relative concentration of HD 1131 Da in ALS-9 is also worthy of note. ALS-9 is where one would expect to find the monomer of HD, and the HD 1131 Da ion mass is the base peak for this gel segment.

HD has been reported to be a component of the axonal cytoskeleton complex that transports brain-derived neurotrophic factor (BDNF, Gauthier et al. 2004), and in the presently described disclosure evidence of possible HD incorporation into ALS ˜30,000 kDa protein aggregates has been found. So, is there evidence that BDNF may also be entwined into these aggregates? One “unique” BDNF tryptic peptide present in gel segment ALS-1 at greater than 20/20 relative intensity/peak area was found, the oxidized methionine peptide of 1523.7621 Da (FIG. S32, not shown). In gel segment ALS-1, the relative intensity of this peptide was 34.57, while the corresponding value in gel segment CON-1 was zero. Having 15 amino acids, the MW of this peptide is highly characteristic of BDNF. Similar data were obtained for a smaller (i.e., not relative intensity/peak area >20/>20) but “unique” BDNF tryptic peptide (1980.9219 Da).

Although ANDR (MW 98.2 kDa) is much smaller than HD, a relatively comparable situation is seen in its tryptic peptide data. The gel lane profile data for ion mass 1732.0095 Da, the only “unique” ANDR peptide present in ALS-1 at >20/>20 relative intensity/area, is illustrated in FIG. S33 (not shown). In ALS-1 this is yet another of the five ion masses present at a concentration exceeding that of trypsin 842. Another ten >20/>20 ALS-1 ion masses were also found for ANDR that were additionally characteristic for at least one other protein investigated in this work (data not shown).

Hence, presently disclosed information suggests that the four largest ion mass peaks in ALS-1 (FIG. S4, not shown), all greater than trypsin 842, consist of peptides characteristic of (1) neurofilament-M and neurofilament-66 (980.5367 Da), (2) ubiquitin conjugation factor E4 B and synaptojanin 2 (1454.8242 Da), (3) huntingtin (2160.2717 Da, and (4) androgen receptor (1732.0095 Da). The fifth ion mass peak in ALS-1 larger than trypsin 842 is at 836.4753 Da. Such relatively small tryptic peptides are frequently found to be characteristic of several parent proteins, and this ion mass is characteristic of ubiquitin-activating enzyme E11, MICAL-2, dystonin and hamartin.

With future work related to Alzheimer's disease (AD) and Parkinson's disease (PD) in mind, the inventor searched the presently described peptide MS database to see if significant amounts of AD- and PD-related tryptic peptides might be detected. As the transgenic G93A hSOD1 murine model of ALS is characterized by a state of oxidative stress, like AD and PD, perhaps there is some evidence in our initial work suggesting that AD- or PD-related tryptic peptides might have been incorporated into the ultra high MW protein aggregates. Indeed, the inventor found evidence that this is the case.

Although β-amyloid peptide 1-40 (Abeta 1-40) and β-amyloid peptide 1-42 (Abeta 1-42) are well known components of AD extracellular senile plaques, it has also been reported that these peptides can be detected inside affected neurons in AD (Lee et al., 1998). Furthermore, evidence has been presented which indicates that intraneuronal Abeta 1-42 and 1-40 accumulation is an early event in AD, preceding the appearance of neurofibrillary tangles (NFTs) or senile plaques (Gouras et al., 2000; Fernandez-Vizarra et al., 2004; Stokin et al., 2005; Billings et al., 2005). With such work in mind, the inventor also examined the presently described G93A hSOD1 murine tryptic peptide MS database for evidence of ion masses characteristic of Abeta peptides. Abeta 1-40 and 1-42 are small proteins, with molecular weights of 4,234 and 4,418 Da, respectively. For each, its SwissProt list of tryptic peptides only has three entries. Yet, as illustrated in FIG. S34 (not shown), the present inventor was able to detect one “unique” ion mass characteristic of each of these two forms of Abeta (1085.6387 Da for Abeta 1-40 and 1269.7598 Da for Abeta 1-42), and gel segment ALS-1 showed relative concentration values that were far higher than those observed in gel segment CON-1.

Presenilin 1 gene mutations are the main cause of early onset autosomal dominant AD (Hanisch and Kolmel, 2004). As illustrated in FIG. S35 (not shown), gel segment 1 ultra high MW aggregates in the ALS protein lane show relatively large amounts of four “unique” peptide ion masses (2149.0593, 2976.5943, 3247.7701 and 3417.6621 Da) characteristic of presenilin 2 (Alzheimer disease 4 homolog, Swiss-Prot file Q61144) and one “unique” peptide ion mass (2951.7803 Da) characteristic of presenilin 1 (Swiss-Prot file P49769). However, gel segment CON-1 shows no detectable amounts of these peptides. All five of these ion masses were present at <20 relative intensity in ALS-1, but their gel lane profiles are still informative. These are large tryptic peptides, ranging from 20 amino acids for ion mass 2149 Da to 29 amino acids for ion mass 3418 Da.

Previous studies on presenilin 1 have described at least two roles for this protein. Besides playing a role in amyloid-beta precursor protein (APP) cleavage, presenilin 1 has also been described as a carrier for cadherin, a protein involved in synaptic cell membrane adhesion (Uemura et al., 2003). So, the present inventor searched the peptide MS database for evidence that cadherin is likewise incorporated into ALS ultra high MW protein aggregates. As illustrated in FIG. S36 (not shown), three “unique” peptide ion masses were found that are characteristic of neural cadherin (1837.9078, 2273.2063 and 3343.6838 Da). One of these ion masses was present in ALS-1 at <20 relative intensity. But these are large ion masses (e.g. ion masses having 18, 21 and 32 amino acids), and their data suggest that this protein may also become incorporated into ultra high MW ALS axonal cytoskeleton protein aggregates. These three peptides could not be detected in CON-1. These preliminary observations suggest that axonal presenilin 1 and its cargo of cadherin might play roles in disease-related loss of synaptic membrane adhesion, and this may contribute to some ALS patients' loss of cognitive abilities.

In order to monitor the electrophoretic migration of some polypeptides involved with the kinesin motor protein complex (i.e., anterograde fast axonal transport away from the nerve cell body), the gel lane profiles of two kinesin proteins were determined. Kamal and coworkers (2001) have proposed that APP acts as a kinesin-I membrane receptor, influencing the axonal transport of presenilin-1 and 6-secretase, and that APP can be hydrolyzed to liberate β-amyloid peptides while participating in anterograde axonal transport. Hence, axonal transport may play a role in the etiology of AD. In the presently disclosed study, the gel lane profiles of two “unique” tryptic peptides characteristic of kinesin heavy chain isoform 5C (Kif5c, ion masses 1500.7904 and 1894.9215 Da) and two “unique” tryptic peptides characteristic of kinesin-like protein (KIF1A, ion masses 902.4842 and 1195.7045 Da) were determined (FIG. S37, not shown). Comparing the data of ALS-1 to that of CON-1, the ALS-1 values appear to be approximately three-times higher.

Studies initially defined by Hattori (2004) have led to the discovery that mutations in parkin, an E3 ubiquitin ligase account for most cases of hereditary PD. Hattori has noted that, as parkin is a ubiquitin ligase and alpha-synuclein fibrils have been shown to inhibit the 26S proteosome, the relationships of these proteins to PD etiology appears to fall within the general theme of abnormal protein accumulation underlying a class of neurodegenerative disorders. Under conditions of oxidative stress, such as found in PD, genetically normal parkin becomes S-nitrosylated at three cysteine residues, and such oxidative damage impairs the ability of this enzyme to ubiquitinate proteins (Chung et al., 2004). There is also some interest in the relationship of tau protein to the etiologies of parkinson-like diseases. The most obvious example here is familial frontotemporal dementia and parkinsonism linked to chromosome 17 (Spillantini and Goedert, 2001). Forms of this disorder are directly based on mutations in the tau gene. In addition, abnormal accumulations of tau protein have been reported in cases of autosomal-recessive juvenile parkinsonism (Mori et al., 1998; van de Warrenburg et al., 2001). As summarized by Spillantini and Goedert (2001), an association between either the AO allele of the tau gene or the H1 halotype of the tau gene and idiopathic PD have been reported. Taking in account the central role played by alpha-synuclein in PD, Spillantini and Goedert (2001) speculated that tau and alpha-synuclein may interact within nerve axons by processes yet to be determined. Tau is now known to be one of the most abundant axonal microtubule-associated proteins (Garcia and Cleveland, 2001). As the transgenic G93A hSOD1 murine model of ALS is characterized by a state of oxidative stress, like PD, perhaps there is some evidence of PD-related peptide incorporation into the ultra high MW axonal cytoskeleton protein aggregates that were studied in the work described herein. Indeed, there is some evidence suggesting that this is the case. Gel lane profile plots for two “unique” tryptic peptides characteristic of E3 ubiquitin-protein ligase parkin (Park2) isoform 1 (983.5268 and 2560.2686 Da) and three “unique” tryptic peptides characteristic of microtubule-associated protein tau (i.e., tau protein; 1305.7385, 1309.7184, and 1768.9374 Da) are illustrated in FIG. S38 (not shown). Looking at the ˜30,000 kDa data, the numbers fall into two clearly defined groups, with the ALS lane showing values about three times higher than those seen for the control lane.

Section 6 Colored Ultra High Molecular Weight Protein Standards Brief Summary. This aspect of the invention embodiment is a product useful as a methodological tool in biomedical research laboratories. Two examples are disclosed herein. One example is based on the chemical modification of human immunoglobulin M [i.e., IgM], and the other example is based on the chemical modification of the myosin H peptide. IgM and myosin H are referred to as starting substances. For the present purposes, each of these starting substances will be referred to as being a monomeric form of the substance. The method of production of this aspect of the invention embodiment consists of two parts. In the first part, a starting substance is combined with a dilute solution of glutaraldehyde for about 10-20 minutes at room temperature. This chemical reaction, a process of covalent polymerization, will convert the monomeric form of the starting substance into a mixture of monomeric, dimeric, trimeric, tetrameric, pentameric, hexameric and higher forms of the monomeric substance. These polymeric forms of the starting substance will include subunits that have been covalently linked to one another. Said covalent chemical linkage will depend primarily on the reaction between primary amine functional groups on the sidechains of lysine residues and glutaraldehyde, producing covalent bond cross-links of the Schiff base variety, or chemical variations thereof. In the second part of the method of production, a mixture of the products derived from part one will be combined with a chemically reactive, small molecular weight dye-containing substance, so as to covalently bind dye molecules to the monomeric, dimeric and polymeric forms of the starting substance. After each part of the method of production, unwanted reagents will be eliminated.

For each starting substance, the final product will be a mixture of the dye-colored derivatives of the monomeric, dimeric and polymeric forms of the starting substance. Said final product can be described as a “protein ladder,” an “ultra high molecular weight protein standard,” a “colored ultra high molecular weight protein standard,” an “ultra high MW protein standard” or a “colored ultra high MW protein standard.” The molecular weight of monomeric myosin H peptide derived from rabbit muscle is 200 kDa. Hence, its corresponding polymerized forms will have molecular weights of 400 kDa, 600 kDa, 800 kDa, 1,000 kDa and possibly higher. The covalent binding of dye molecules to said peptides will increase their molecular weights only nominally. The molecular weight of the native human IgM molecule complex, which includes several individual peptides, is 970 kDa. Hence, its corresponding polymerized forms will have molecular weights of 1,940 kDa, 2,910 kDa, 3,880 kDa, 4,850 kDa and possibly higher. Here too, the covalent binding of dye molecules to said IgM peptide complexes will increase their molecular weights only nominally. In the case of IgM, the effect of glutaraldehyde treatment will be to both covalently bind the individual peptides of the native complex to one another and to bind some IgM monomeric complexes to one another.

Applications/Commercial use/Products envisioned. The “protein ladders” of this aspect of the invention embodiment will be of use in protein purification laboratory procedures and protein analytical laboratory procedures based on use of sodium dodecyl sulphate/polyacrylamide gel electrophoresis (i.e., SDS/PAGE), agarose gel electrophoresis, composite agarose-polyacrylamide gel electrophoresis of the instant disclosure and column chromatography based on use of resins which separate proteins according to size (e.g., Sepharose 4B as per Payne [1973]). In particular, said “protein ladder” products of the instant disclosure can be used in the analysis of experimental protein samples of biological origin wherein the experimental proteins of interest are of molecular weights in excess of 200 kDa.

As presently disclosed by way of examples, the inventor illustrates the process of making a “protein ladder” product based on polymerization of IgM and a second “protein ladder” product based on myosin polymerization. The myosin “protein ladder” product can serve as a set of visible protein standard bands on SDS/PAGE gels of, for example, 3.5% to 5.0% cross-linked acrylamide. Such SDS/PAGE gels can separate mixtures of proteins of biological origin in the molecular weight range of approximately 200 kDa to 800 kDa. Also, the IgM “protein ladder” product can serve as a set of visible protein standard bands on agarose gels of, for example, 1.0% to 1.5% SeaKem® Gold agarose. Such agarose gels can separate mixtures of proteins of biological origin in the molecular weight range of approximately 600 kDa to 5,000 kDa. Alternatively, the IgM “protein ladder” product can serve as a set of visible protein standard bands on composite agarose-polyacrylamide gels of the instant disclosure as described above in Section 1 of the DETAILED DESCRIPTION OF THE INVENTION.

As regards the applications and commercial uses of these products, this inventor makes the following observation. During the past ten years, the field of proteomics has become well established. Indeed, this field has become an industry in its own right. Many large drug companies have entire departments that just do this work. Proteomics research is based on the technology of two-dimensional gel electrophoresis. In such protocols, a complex mixture of proteins is separated along one axis according to isoelectric point (i.e., net molecular charge), then separated along a perpendicular axis according to molecular size. As far as this inventor knows, proteomics studies have not considered proteins having molecular weights >200 kDa. One reason why this may be so is that presently the technology of the instant disclosure, including “protein ladder” colored high molecular weight standards, is not commercially available. Once the presently disclosed technology does become available to scientists in the proteomics research field, the field of proteomics research may be expanded significantly.

The colored high molecular weight protein standards described herein may be of use for biomedical scientists studying of variety subjects in the field of analytical protein chemistry. A sampling of research subjects involving the study of high molecular weight proteins includes: (1) protein aggregates characteristic of neurodegenerative diseases, such as amyotrophic lateral sclerosis, Alzheimer's disease, Parkinson's disease and Huntington's disease; (2) plectin, an axonal cytoskeleton protein of MW>500 kDa; (3) protein cross-linkage issues related to diabetes; (4) blood platelet protein analysis, e.g., the study of von Willebrand factor multimers or the study of multimerin multimers; (5) dystrophin, MW 400 kDa; (6) low density lipoprotein receptor-related protein peptide MW 515 kDa; (7) protein aggregation resulting from the induction of oxidative stress; and (8) the 26S (i.e., 1,500 kDa) ubiquitin-proteosome proteolytic complex. The study of such subjects has heretofore been impeded by the lack of the convenient, cost effective laboratory products needed to assure the success of such efforts. The “protein ladder” colored high molecular weight standards of the instant disclosure can provide scientists with key research tools needed to facilitate their studies.

Closest known similar technology or competing products. The idea of using dilute solutions of glutaraldehyde to partially cross-link a protein so as to obtain a series of progressively larger polymers has already been disclosed in the prior art. Prior art disclosures of this kind include: (1) Richards and Knowles (1968); (2) Darawshe and Daniel (1991); (3) Payne (1973); (4) Furlan and Beck (1975); (5) Fass et al. (1978); (6) Azem et al. (1995); and (7) Carden and Eagles (1983). None of these publications discloses the use of glutaraldehyde to selectively polymerize the myosin H monomeric peptide, although the in vitro cross-linking of rabbit myosin subfragment 1 by use of malonaldehyde has been described by Li and King (1997). Rabbit myosin subfragment 1 has a molecular weight of 95 kDa, and is a subunit of native myosin distinct from rabbit myosin H monomeric peptide (MW 200 kDa; Sigma Chemical Co., catalog number MK-7659]. Fass et al. (1978) described the in vitro polymerization of native human IgM, and the method of Fass et al. (1978) was used and further illustrated by Ruggeri and Zimmerman (1980). Yet none of these examples of prior art has anticipated or particularly disclosed either the method of preparation of the instant dye-colored ultra high MW protein polymers or the utility of such dye-colored ultra high MW protein polymers.

Pierce Chemical Company sells N-hydroxysuccinimide-fluorescein (i.e., NHS-fluorescein, catalog number 46100). Pierce provides for its customers document number 0367w (i.e., “Instructions NHS-Fluorescein”), which describes the utility of this chemical entity as a fluorescent dye capable of reacting with and covalently binding to various proteins. Page 3 of document number 0367w includes a text section entitled “Sample protocol for labeling IgG with NHS-fluorescein.” However, this text section does not teach the in vitro labeling of immunoglobulin polymers. Pierce also sells “EZ-Label™ Fluorescein Protein Labeling Kit” (catalog number 53000), which embodies a particular method of use of NHS-fluorescein for the general labeling of proteins. Pierce also provides for its customers document number 1323w (i.e., “Instructions EZ-Label™ Fluorescein Protein Labeling Kit), which teaches use of the kit. But, here too, document number 1323w does not teach the in vitro labeling of immunoglobulin polymers or the in vitro labeling of any other protein polymers.

Pierce Chemical Company also sells N-hydroxysuccinimide-rhodamine (i.e., NHS-rhodamine, catalog number 46102). Pierce provides for its customers document number 0366 (i.e., “Instructions NHS-Rhodamine”), which describes the utility of this chemical entity as a fluorescent dye capable of reacting with and covalently binding to various proteins. Page 2 of document number 0366 includes a text section entitled “Sample protocol for labeling. IgG with NHS-rhodamine.” However, this text section does not teach the in vitro labeling of immunoglobulin polymers. Pierce also sells “EZ-Label™ Rhodamine Protein Labeling Kit” (catalog number 53002), which embodies a particular method of use of NHS-rhodamine for the general labeling of proteins. Pierce also provides for its customers document number 1321w (i.e., “Instructions EZ-Label™ Rhodamine Protein Labeling Kit), which teaches use of the kit. But, here too, document number 1321w does not teach the in vitro labeling of immunoglobulin polymers or the in vitro labeling of any other protein polymers.

Presently, no commercial source exists for the products disclosed herein. Several companies currently sell colored standard protein mixtures useful as visual reference tools in the performance of SDS/PAGE. These products are well known, and virtually every laboratory in which analytical protein chemistry takes place has such products on hand. However, each of these products includes a combination of proteins covering a molecular weight size range of smaller proteins. For example, the “MultiMark Multi-Colored Standard” (Invitrogen Life Technologies, catalog number LC5725) includes a colored mixture of nine standard proteins ranging in size from insulin (4 kDa) to myosin H monomer (250 kDa). Pierce Chemical Company sells the “TriChromRanger Prestained Protein Molecular Weight Marker Mix” (catalog number 26689), which includes a colored mixture of seven standard proteins ranging in size from lysozyme (15.8 kDa) to myosin H monomer (209 kDa). Bio-Rad Laboratories sells the “Kaleidoscope Polypeptide Standards” mixture (catalog number 161-0324), which includes a multi-colored mixture of seven standard proteins ranging in size from aprotinin (7.6 kDa) to myosin H monomer (216 kDa). Bio-Rad also sells “Prestained SDS-PAGE Standards, high range” (catalog number 161-03090, which includes four blue stained proteins ranging in size from ovalbumin (48 kDa) to myosin H monomer (204 kDa). For all such presently existing products that this inventor is aware of, the largest peptide is the myosin H monomer, its apparent size varying somewhat from one product to another based on the extent to which various dyes are bound.

Hence, for scientists wishing to do analytical protein chemistry studies based on the examination of proteins or protein complexes larger than the myosin H monomer, no commercially available prestained mixtures of appropriate protein standards currently exist.

Differences and advantages over other technology or products. The present technology is advantageous in that it facilitates the study of proteins and protein complexes >200 kDa. Having such ultra high MW “protein ladder” colored protein standard products commercially available will permit scientists to conveniently examine aspects of molecular biology that at present are not readily approachable, e.g., the estimation of ultra high MW protein aggregate size. Having such new products available will additionally permit scientists to visually monitor the status of ultra high MW analytical protein separations as such studies are in progress, providing real time information about the success of experiments and offering the opportunity to modify experimental conditions according to a scientist's preferences while experiments are in progress.

### Example Number One

Human IgM polymerization. A series of reactions is done, each in a final reaction volume of 100 ul. Each reaction will have the same ingredients. Do time points at zero, 5 min, 10 min, 15 min, 20 min, 25 min and 30 min. Each reaction mixture will contain 100 μg protein (i.e., 1.0 mg/ml) and 0.2% glutaraldehyde (final concentration) in 10 mM NaPO₄ buffer, pH 7.0, incubated at room temperature. Terminate each reaction by the addition of an equal volume of 2% SDS (as per Payne [1973]), then put reaction mixtures on ice.

In order to stabilize and preserve any Schiff base cross-links that have been formed, the terminated reaction mixtures are reduced with 50 mM NaCNBH₃ (Esterbauer and Cheeseman, 1990). Weigh out and immediately add 32 mg of NaCNBH₃ into 5 ml of 10 mM NaPO₄ buffer, pH 7.0 (kept on ice). Then add 200 μl of this stock NaCNBH₃ solution to each 2% SDS-terminated 200 μl protein cross-linking reaction (i.e., final concentration 50 mM NaCNBH₃). These terminated reaction mixtures are then put onto a vertical wheel rotator for 4 hours at room temperature.

Each terminated reaction mixture is then dialyzed against 10 mM NaPO₄ buffer, pH 7.0 for 24 hours.

Run 20 μg protein samples on an electrophoresis gel system. On each gel, run Invitrogen MultiMark Multi-Colored Standard in one or more lanes. For each gel, run the blue myosin band down to just a few millimeters above the lower edge of the gel. Stain the gel with Pierce GelCode SilverSNAP Stain Kit in order to visualize the resolution of the “protein ladder” gel band pattern.

Observing the time-dependent polymerization results, choose an incubation time that gives a relatively even mixture of monomer, dimer and several polymers of higher molecular weight covering the desired molecular weight range. With the optimized conditions for protein cross-linking now defined, the investigator proceeds to examine the process of protein labeling with Pierce NHS-rhodamine. Pierce sells “EZ-Label™ Rhodamine Protein Labeling Kit” (catalog number 53002), which embodies a particular method of use of NHS-rhodamine for the general labeling of proteins. Pierce also provides for its customers document number 1321w (i.e., “Instructions EZ-Label™ Rhodamine Protein Labeling Kit), which teaches use of the kit. Document number 1321w does not teach the in vitro labeling of immunoglobulin polymers or the in vitro labeling of any other protein polymers. But it is the instant inventor's understanding that the Pierce “EZ-Label™ Rhodamine Protein Labeling Kit” provides a convenient starting point for protein polymer labeling that can be readily adapted by one of ordinary skill in the art.

Dye labeling incubation reactions will be for time zero, 15 min, 30 min, 45 min, 60 min, 90 min and 120 min. Also, one reaction will have no NHS-rhodamine. Run 20 μg protein samples on an appropriate gel system (as determined above). Observe the colored protein pattern apparent to the eye upon completion of the electrophoresis; scan a digital image of this. Then, develop the gel with the Pierce GelCode SilverSNAP Stain Kit, so as to reveal the position of the polymerized protein bands in the lane(s) having sample that was not treated with NHS-rhodamine. Scan the gel image again. By comparing the relative position of a particular protein band (e.g., dimer or trimer) having no bound dye to the position of the corresponding protein band with bound dye, the degree to which the binding of dye has altered the MW of the cross-linked protein will become apparent. Select a dye incubation time that, on one hand, gives protein labeling readily apparent to the eye and, on the other hand, only has a minimum effect on the apparent MW of the polymer (i.e., as compared to its cross-linked counterpart lacking bound dye).

The size (i.e., scale) of the preferred starting substance cross-linking reaction and colored dye labeling reaction can be increased so as to provide final “colored ultra high MW protein standard” on a commercially useful basis.

### Example Number Two

Myosin polymerization. Perform a corresponding series of cross-linking reactions and dye labeling reactions starting with the heavy polypeptide of native myosin.

Variations on the Method of “Protein Ladder” Synthesis. In variations of the two examples noted above that fall within the metes and bounds of this disclosure, the protein monomer (i.e., starting substance) can be different (e.g., lysozyme as per Payne [1973], carboxypeptidase-A as per Richards and Knowles [1968], fibrinogen as per Furlan and Beck [1975], or bovine serum albumin as per Lederer and Klaiber [1999] or Mikulíková et al. [2005]), the aldehyde reagent used for protein cross-linking can be different (e.g., 4-hydroxy-2-nonenal, 4-oxo-2-nonenal and acrolein as per Sayre et al. [2006], with NaBH₄-reduction used instead of NaCNBH₃ reduction; or glyoxal or methylglyoxal as per Lederer and Klaiber [1999]), the aldehyde reagent concentration used for protein cross-linking can be different (e.g., from 0.03 mg glutaraldehyde/ml [i.e., 0.003%] to 2.0 mg glutaraldehyde/ml [i.e., 0.2%], as per Furlan and Beck [1975]), the buffer used for cross-linking reactions can be different (e.g., 0.1 M NaCl/0.05 M Tris-HCl buffer, pH 7.3 as per Furlan and Beck [1975]), the incubation time for the protein cross-linking reaction can be different (e.g., 24 hours as per Payne [1973]) or one week as per Lederer and Klaiber [1999]), the incubation temperature can be different (e.g., 37° C. as per Lederer and Klaiber [1999]), the cross-linking reaction can additionally contain from 5% to 50% glycerol, the reagent(s) used to terminate the cross-linking reaction can be different (e.g., glycine, alanine or lysine addition to a final concentration of 0.05 M), the colored dye bound to the cross-linked “protein ladder” mixture can be different (e.g., Pierce NHS-fluorescein), and the reaction conditions used for binding the colored dye to the cross-linked “protein ladder” can be different (e.g., differences in incubation time, incubation buffer or incubation temperature). In further variations of these two examples that fall within the metes and bounds of this disclosure, the final colored “protein ladder” cross-linked substances can be resuspended in a buffer containing from 5% to 50% glycerol (v/v basis) or a buffer consisting of 1% sodium dodecyl sulphate in 6 M urea, 0.025 M sodium phosphate, pH 7.1, so as to ensure that the final product remains uniformly suspended in a liquid form. In a further variation of these two examples that falls within the metes and bounds of this disclosure, two or more “protein ladder” mixtures, each having a differently colored dye bound to protein polymers, can be combined. For purposes of this disclosure, any such composition noted above in this section is defined as consisting of a mixture of purified, covalently cross-linked, dye-colored proteins of a multimeric series of standard proteins.

### REFERENCES

A. T. Andrews, Electrophoresis: Theory, Techniques, and Biochemical and Clinical Applications (Clarendon, Oxford, 1981), pp. 106-127.
D. M. Appelt, B. J. Balin, Brain Res. 745, 21 (1997).
C. S. Atwood et al., Biochemistry 43, 560 (2004).
A. Azem, I. Shaked, J. P. Rosenbusch, E. Daniel, Biochim. Biophys. Acta 1243 151 (1995).
M. Baker et al., Nature 442, 916 (2006).
L. Bertram et al., New Engl. J. Med. 352, 884 (2005).
L. M. Billings et al., Neuron 45, 675 (2005).
M. J. Carden, P. A. Eagles, Biochem. J. 215 227 (1983).
Y. Z. Chen et al., Am. J. Hum. Genet. 74, 1128-35 (2004).
K. K. Chung et al., Science 304, 1328 (2004).
R. B. Counts, S. L. Paskell, S. K. Elgee, J. Clin. Invest 62, 702 (1978).
M. Cruts et al., Nature 442, 920 (2006).
R. Daniel, E. Daniels, Z, He, A. Bateman, Dev Dyn. 227, 593 (2003).
S. Darawshe, E. Daniel, Eur. J. Biochem. 202 169 (1991).
R. Dei et al., Acta Neuropathol. 104, 113 (2002).
M. D. Diaz-Hernández et al., J. Neurosci. 24, 9361 (2004).
H. Doi et al., FEBS Lett. 571, 171 (2004).
K. B. Elkon, P. W. Jankowski, J. L. Chu, Anal. Biochem. 140, 208 (1984).
H. Esterbauer, K. H. Cheeseman, Methods Enzymol. 186, 407 (1990).
D. N. Fass, G. J. Knutson,E. J. Walter Bowie, J. Lab. Clin. Med. 91 307 (1978).
F. Fenaille et al., J. Am. Soc. Mass Spectrom. 14, 215 (2003).
P. Fernandez-Vizarra et al., Histol. Histopathol. 19, 823 (2004).
R. J. Ferrante et al., Ann. Neurol. 42, 326 (1997).
K. Fujita et al., J. Neurosci. Res. 45, 276 (1996).
M. Furlan, E. A. Beck, Thromb. Res. 7 827 (1975).
C. Gabelli, D. G. Stark, R. E. Gregg, H. B. Brewer, Jr, J. Lipid Res. 27, 457 (1986).
M. L. Garcia, D. W. Cleveland, Current Opin. Cell Biol. 13, 41 (2001).
L. R. Gauthier et al., Cell 118, 127 (2004).
G. K. Gouras et al., Am. J. Pathol. 156, 15 (2000).
A. J. Grierson, G. V. Johnson, C. C. Miller, Neurosci. Lett. 298, 9 (2001).
S. Gunawardena et al., Neuron 40, 25 (2003).
E. D. Hall et al., J. Neurosci. Methods 76, 115 (1997).
E. D. Hall et al., J. Neurosci. Res. 53, 66-77 (1998).
F. Hanisch, H. W. Kolmel, Eur. J. Med. Res. 9, 361 (2004).
N. Hattori, Rinsho Shinkeigaku 44, 241 (2004).
C. P. M. Hayward, T. E. Warkentin, P. Horsewood, J. G. Kelton, Blood 77, 2556 (1991).
R. Heimer, P. M. Sampson, Anal. Biochem. 162, 330 (1987).
D. Heinegard, Y. Sommarin, E. Hedbom, J. Wieslander, B. Larsson, Anal. Biochem. 151, 41 (1985).
A. Kamal et al., Nature. 414, 643 (2001).
I. G. Kirkinezos et al., J. Neurosc. 25, 164 (2005).
S. Knox, J Melrose, J. Whitelock, Proteomics 1, 1534 (2001).
F. Lasne, O. Benzerara, Y. Lasne, Anal. Biochem. 132, 338 (1983).
M. O. Lederer, R. G. Klaiber, Bioorg. Med. Chem. 7 2499 (1999).
S. J. Lee et al., Nature Med. 4, 730 (1998).
S. J. Li, A. J. King, J. Agric. Food Chem. 47 3124 (1997).
L. Liao et al., J. Biol. Chem. 279, 37061 (2004).
Q. Liu et al., Mol. Aspects. Med. 24, 305 (2003).
J. Liu et al., Neuron 43, 5 (2004).
D. B. Lubahn, L. M. Silverman, Clin. Chem. 30, 1689 (1984).
A. Lunkes et al., Mol. Cell. 10, 259 (2002).
I. R. Mackenzie et al., Ann. Neurol. 61, 427 (2007).
N. Maeda et al., J. Biol. Chem. 266, 1109 (1991).
A. L. Mah, G. Perry, M. A. Smith, M. J. Monteiro, J. Cell Biol. 151, 847 (2000).
C. A. McDevitt, H. Muir, Anal. Biochem. 44, 612 (1971).
J. Melrose, C. B. Little, P. Ghosh, Anal. Biochem. 256, 149 (1998).
M. J. Melrose, S. Smith, P. Ghosh, J. Anat. 197, 189 (2000).
T. Miki, Y. Orii, Anal. Biochem. 146, 28 (1985).
K. Mikulíková, I. Miksik, Z. Deyl, J. Chromatogr. B Analyt. Technol. Biomed. Life Sci. 815 315-31 (2005).
C. C. Miller, B. H. Anderton, J. Neurochem. 46, 1912 (1986).
H. Mori, Neurology 51, 890 (1998).
C. Mottley, R. E. Robinson, R. P. Mason, Arch. Biochem. Biophys. 289, 153 (1991).
M A. Norlund, J. M. Lee, G. M. Zainelli, N. A. Muma, Brain Res. 851, 154 (1999).
P. Orban, R. S. Devon, M. R. Hayden, B. R. Leavitt, Handb. Clin. Neurol. 82, 301 (2007).
Z. Y. Park, D. H. Russell, Anal. Chem. 73, 2558 (2001).
J. W. Payne, Biochem. J. 135 867 (1973).
A. C. Peacock, C. W. Dingman, Biochemistry 7, 668 (1968).
W. A. Pedersen et al., Ann. Neurol. 44, 819-824 (1998).
S. Peats, Anal. Biochem. 140, 178 (1984).
B. A. Perret, M. Furlan, E. A. Beck, Biochim. Biophys. Acta 578, 164 (1979).
G. Perry, BioMetals. 16, 77 (2003).
F. M. Richards, J. R. Knowles, J. Mol. Biol. 37 231 (1968).
P. G. Righetti, J. Biochem. Biophys. Methods 19, 1 (1989).
J. Robertson et al., Neurosci. Lett. 420, 128 (2007).
L. Rossi, M. F. Lombardo, M. R. Ciriolo, G. Rotilio, Neurochem. Res. 29, 493 (2004).
Z. M. Ruggeri, T. S. Zimmerman, J. Clin. Invest. 65 1318-1325 (1980).
L. M. Sayre, D. Lin, Q. Yuan, X. Zhu, X. Tang, Drug Metab. Rev. 38 651 (2006).
J. C. Schymick et al., J Neurol Neurosurg Psychiatry 78, 754 (2007).
D. J. Selkoe, C. Abraham, Y. Ihara, Proc. Natl. Acad. Sci. U.S.A. 79, 6070 (1982).
S. M. Singer et al., Neurochem. Int 40, 17 (2002).
K. Sleegers et al., Neurology 71, 253 (2008).
M. G. Spillantini, M. Goedert, JAMA 286, 2324 (2001).
T. H. Steinberg et al., Electrophoresis 21, 486 (2000).
G. B. Stokin et al., Science 307, 1282 (2005).
M.-H. Suh et al., Anal. Biochem. 343, 166 (2005).
G. Szebenyi et al., Neuron 40, 41 (2003).
K. Uemura et al., J. Neurosci. Res. 74, 184 (2003).
B. P. van de Warrenburg et al., Neurology 56, 555 (2001).
J. B. Varelas, N. R. Zenarosa, C. J. Froelich, Anal. Biochem. 197, 396 (1991).
M. P. Vitek et al., Proc. Natl. Acad. Sci. U.S.A. 91, 4766 (1994).
T. Wataya et al., J. Biol. Chem. 277, 4644 (2002).
A. Yamamoto, J. J. Lucas, R. Hen, Cell 101, 57 (2000).
X. Zhu, M. A. Smith, G. Perry, G. Aliev, Am. J. Alz. Dis. Other Dementias. 19, 345 (2004).
   Without further elaboration the foregoing will so fully illustrate this invention so that others may, by applying current or future knowledge, adapt the same for use under various conditions of service.

I claim:

1) A method for the molecular characterization of ultra high molecular weight nerve protein aggregates characteristic of a neurodegenerative disease based on isolation thereof from an animal nerve tissue homogenate by use of composite agarose-polyacrylamide gel electrophoresis and subsequent peptide mass spectrometry.

2) The method of claim 1) wherein the neurodegenerative disease is selected from the closed group consisting of human familial or non-familial Charcot-Marie-Tooth disease, Alzheimer's disease, Parkinson's disease, diseases based on expansions in tandem DNA repeats, spinal muscular atrophy, Friedreich's ataxia, giant axon neuropathy, juvenile ceroid-lipofuscinosis, amyotrophic lateral sclerosis, diabetic polyneuropathy and Down's syndrome; as well as transgenic non-human animal models corresponding to a previously stated human neurodegenerative disease and gene knockout non-human animal models corresponding to a previously stated human neurodegenerative disease.

3) The method of claim 1) wherein the composite agarose-polyacrylamide gel contains from 0.1% agarose to 5.0% agarose.

4) The method of claim 1) wherein the composite agarose-polyacrylamide gel contains from 0.5% polyacrylamide to 6.0% polyacrylamide.

5) The method of claim 1) wherein the ultra high molecular weight protein aggregates are of sizes within the molecular weight range of from approximately 225 kDa to approximately 100,000 kDa.

6) The method of claim 1) wherein the endopeptidase used to prepare an excised composite agarose-polyacrylamide gel segment for mass spectrometry analysis is selected from the closed group consisting of trypsin, chymotrypsin, endoproteinase Arg-C, carboxypeptidase Y optionally in combination with carboxypeptidases A and B, endoproteinase Asp-N, endoproteinase Glu-C and endoproteinase Lys-C.

7) The method of claim 1) wherein the peptides derived from endopeptidase treatment of isolated ultra high molecular weight protein aggregates separated on and embedded within a composite agarose-polyacrylamide gel subsequent to electrophoretic resolution are subjected to mass spectrometry characterization by use of a matrix-assisted laser desorption/ionization—time of flight mass spectrometer, a matrix-assisted laser desorption/ionization-time-of-flight/time-of-flight tandem mass spectrometer, an electrospray ionization tandem mass spectrometer, a hybrid linear ion trap-Fourier transformion cyclotron resonance mass spectrometer, a linear ion trap mass spectrometer, an ETD-enabled hybrid linear ion trap-orbitrap mass spectrometer or a quadrupole ion trap mass spectrometer.

8) The method of claim 7) wherein the mass spectrometry data is used to prepare a database that defines the unique molecular characteristics of an ultra high molecular weight protein aggregate derived from homogenized animal nerve tissue.

9) The method of claim 8) wherein the mass spectrometry databases derived from the analysis of two or more ultra high molecular weight protein aggregates are compared to one another so as to partially characterize the molecular events occurring in the etiological process of a neurodegenerative disease.

10) The method of claim 8) wherein the mass spectrometry databases derived from the analysis of two or more ultra high molecular weight protein aggregates are compared to one another so as to partially characterize the molecular events occurring in the study of a candidate therapeutic drug agent.

11) The method of claim 1) wherein the optical densities of two or more ultra high molecular weight protein aggregates on composite agarose-polyacrylamide gel are compared to one another so as to partially characterize the molecular events occurring in the study of a candidate therapeutic drug agent.

12) The method of claim 1) wherein a nerve tissue ultra high molecular weight protein aggregate metabolic marker characteristic of the presence of a neurodegenerative disease is obtained.

13) The method of claim 12) wherein the nerve tissue ultra high molecular weight protein aggregate metabolic marker is used to monitor the pathological, i.e., clinical, stage of a neurodegenerative disease.

14) The method of claim 12) wherein the nerve tissue ultra high molecular weight protein aggregate metabolic marker is used as a new and novel disease-specific antigen.

15) The method of claim 14) wherein the new and novel disease-specific antigen is used to produce a disease-specific monoclonal antibody or polyclonal antibodies.

16) The method of claim 15) wherein the disease-specific monoclonal antibody or polyclonal antibodies is/are used as the basis for a diagnostic test.

17) The method of claim 16) wherein the diagnostic test is used to identify the presence of sub-clinical amounts of disease-specific antigen(s) in human patient or other animal blood.

18) The method of claim 16) wherein the diagnostic test is used to identify the presence of sub-clinical amounts of disease-specific antigen(s) in human patient or other animal urine.

19) The method of claim 15) wherein the disease-specific monoclonal antibody or polyclonal antibodies is/are used as the basis for tissue screening histological studies.

20) The method of claim 1) wherein the nerve tissue homogenate is derived from a human or other animal tissue sample obtained at autopsy.

21) A composition consisting of ultra high molecular weight protein aggregates derived from homogenized animal nerve tissue separated on and embedded within a composite agarose-polyacrylamide gel subsequent to electrophoretic resolution.

22) A composition consisting of the peptides derived from endopeptidase treatment of isolated ultra high molecular weight protein aggregates derived from homogenized animal nerve tissue separated on and embedded within a composite agarose-polyacrylamide gel subsequent to electrophoretic resolution.

23) A method for obtaining an in-gel Western blot film image of proteins electrophoretically separated on a composite agarose-polyacrylamide gel by the placement of a piece of x-ray film negative in immediate proximity to the gel in such a manner that one of the two large area surfaces of the gel and the x-ray film negative are separated by a thin sheet of clear colorless plastic wrap or a thin sheet of colorless glass.

24) A device useful for obtaining an in-gel Western blot film image of proteins electrophoretically separated on a composite agarose-polyacrylamide gel; wherein said device consists of a piece of x-ray film, a sheet of non-translucent Manila folder paper, a thin sheet of clear colorless plastic wrap or a thin sheet of colorless glass, a composite agarose-polyacrylamide gel, and a rigid plastic sheet or glass sheet of approximately 8 inches width by approximately 8 inches length and of height approximately one-eight inch wherein said items are positioned one on top of the other in said order with the x-ray film on top in one corner of a flat tray of approximately 8½ inches width by approximately 12 inches length and with sides of approximately one-half inch in height.

25) A method for producing a visible pattern of optical density of electrophoretically resolved ultra high molecular weight protein aggregates and ultra high molecular weight proteins on a composite agarose-polyacrylamide gel subsequent to use in an in-gel Western blot film imaging procedure
consisting of subsequently rinsing the gel with water or a buffer, then storing the gel in water or a buffer for at least three days.

26) A composition consisting of a composite agarose-polyacrylamide gel subsequent to use in an in-gel Western blot film imaging procedure has been subsequently rinsed with water or a buffer, then stored in water or a buffer for at least three days, which accordingly reveals a visible pattern of electrophoretically resolved ultra high molecular weight protein aggregates and ultra high molecular weight proteins.

27) A method of producing a mixture of purified, covalently cross-linked, dye-colored proteins of a multimeric series having a size range within approximately 225 kDa to approximately 100,000 kDa wherein each individual component thereof is of an approximately known molecular weight size; said method consisting essentially of chemical polymerization of a starting substance with an aldehyde-containing chemical under conditions fostering the covalent polymerization of said starting substance, followed by a further chemical reaction step that results in the covalent attachment of a dye-colored reagent to the polymerized starting substance.

28) A composition consisting of a mixture of purified, covalently cross-linked, dye-colored proteins of a multimeric series having a size range within approximately 225 kDa to approximately 100,000 kDa wherein each individual component thereof is of an approximately known molecular weight size.

29) A method for estimating the approximate molecular weight of an ultra high molecular weight protein or an ultra high molecular weight protein aggregate by electrophoretic resolution on a composite agarose-polyacrylamide gel with concomitant application to a separate gel sample loading well of a mixture of purified, covalently cross-linked, dye-colored proteins of a multimeric series having a size range within approximately 225 kDa to approximately 100,000 kDa wherein each individual component thereof is of an approximately known molecular weight size.
